SEQUENCE LISTING (6 sequences)

SEQ ID NO: 1 - DNA, 1568 bp, Salmonella choleraesuis
ctaacgatcgggaggtggcgggtccggaggtggttctggtggaggttctatcatggcacaagtcattaatacaaacagcctgtcgctgttgacccagaataacctgaacaaatcccagtctgctctgggtaccgctatcgagcgtctgtcttccggtctgcgtatcaacagcgcgaaagacgatgcggcaggtcaggcgattgctaaccgtttcaccgcgaacatcaaaggtctgactcaggcttcccgtaacgctaacgacggtatttctattgcgcagaccactgaaggcgcgctgaacgaaatcaacaacaacctgcagcgtgtgcgtgaactggcggttcagactgctaacagcaccaactcccagtctgacctcgactccatccaggctgaaatcacccagcgtctgaacgaaatcgaccgtgtatccggtcagactcagttcaacggcgtgaaagtcctggcgcaggacaacactctgaccatccaggttggtgccaacgacggtgaaactatcgatatcgatctgaagcagatcaactctcagaccctgggcctagatacgctgaatgtgcagaaaaaatatgatgtgagcgatactgctgtagctgcttcctattccgactcgaaacagaatattgctgttcctgataaaacagctattactgcaaaaattggtgcagcaaccagtggtggtgctggtataaaagcagatattagctttaaagatggcaagtattacgcgactgtcagtggatacgatgatgccgcagatacagataaaaatggaacctatgaagtcactgttgccgcagatacaggagcagttacttttgcgactacaccaacagtggttgacttaccaactgatgcaaaagcagtttcaaaagttcaacagaatgatactgaaatagcagcaacaaatgcgaaagctgcattaaaagctgcaggagttgcagatgcagaagctgatacagctactttagtgaaaatgtcttatacagataataatggcaaagttattgatggtgggttcgcatttaagacctccggtggttattatgcagcatctgttgataaatctggcgcagctagcttgaaagttactagctacgttgacgctaccactggtaccgaaaaaactgctgcgaataaattaggtggcgcagacggtaaaaccgaagttgttactatcgacggtaaaacctacaatgccagcaaagccgctgggcacaacttcaaagcacagccagagctggcggaagcggctgctacaaccactgaaaacccgctgcagaaaattgatgctgctttggcgcaggtggatgcgctgcgttctgacctgggtgcggttcagaaccgtttcaactccgctatcaccaacctgggcaataccgtaaataacctgtcttctgcccgtagccgtatcgaagattccgactacgcgaccgaagtttccaacatgtctcgcgcgcagattctgcagcaggccggtacctccgttctggcgcaggcgaaccaggttccgcaaaacgtcctctctttactgcgttaagcatgccatg

SEQ ID NO: 2 - DNA, 732 bp, Artificial Sequence
  Description of Artificial Sequence Synthetic light chain construct
catgctcgagtcagagatggagacagacagactcctgctatgggtgctgctgctctgggtgccaggctccactggtgacactgtactgacccagtctcctgctttggctgtgtctccaggagagagggttaccatctcctgtagggccagtgacagtgtcagtacacttatgcactggtaccaacagaaaccaggacagcaacccaaactcctcatctatctagcatcacacctagaatctggggtccctgccaggttcagtggcagtgggtctgggacagacttcaccctcaccattgatcctgtggaggctgatgacactgcaacctattactgtcagcagagttggaatgatccgtggacgttcggtggaggcaccaagctggaattgaaacgggctgatgctgcaccaactgtatctatcttcccaccatccacggaacagttagcaactggaggtgcctcagtcgtgtgcctcatgaacaacttctatcccagagacatcagtgtcaagtggaagattgatggcactgaacgacgagatggtgtcctggacagtgttactgatcaggacagcaaagacagcacgtacagcatgagcagcaccctctcgttgaccaaggctgactatgaaagtcataacctctatacctgtgaggttgttcataagacatcatcctcacccgtcgtcaagagcttcaacaggaatgagtgttagaccctccggacatg

SEQ ID NO: 3 - DNA, 1587 bp, Artificial Sequence
  Description of Artificial Sequence Synthetic heavy chain construct
ggagcccagtcctggactctgaggttctcccactcagtaatcagtactgaagcactgcacagactcctcaccatggacatcaggctcagcttggttttccttgtccttttcataaaaggtgtccagtgtgaagtgcagctggtggagtctggcggaggcttagtacagcctggaaggtccctgaaactctcctgtgcagcctcaggattcactttcagtgactataacatggcctgggtccgccaggctccaaagaagggtctggagtgggtcgcaaccattatttatgatggtagtaggacttactatcgagactccgtgaagggccgattcactatctccagagataatgcaaaaagcaccctatacctgcaaatggacagtctgaggtctgaggacacggccacttattactgtgcaacaaaccgatggttattattacactactttgattactggggccaaggagtcatggtcacagtctcctcagctgaaacaacagccccatctgtctatccactggctcctggaactgctctcaaaagtaactccatggtgaccctgggatgcctggtcaagggctatttccctgagccagtcaccgtgacctggaactctggagccctgtccagcggtgtgcacaccttcccagctgtcctgcagtctggactctacactctcaccagctcagtgactgtaccctccagcacctggtccagccaggccgtcacctgcaacgtagcccacccggccagcagcaccaaggtggacaagaaaattgtgccaagggaatgcaatccttgtggatgtacaggctcagaagtatcatctgtcttcatcttccccccaaagaccaaagatgtgctcaccatcactctgactcctaaggtcacgtgtgttgtggtagacattagccagaatgatcccgaggtccggttcagctggtttatagatgacgtggaagtccacacagctcagactcatgccccggagaagcagtccaacagcactttacgctcagtcagtgaactccccatcgtgcaccgggactggctcaatggcaagacgttcaaatgcaaagtcaacagtggagcattccctgcccccatcgagaaaagcatctccaaacccgaaggcacaccacgaggtccacaggtatacaccatggcgcctcccaaggaagagatgacccagagtcaagtcagtatcacctgcatggtaaaaggcttctatcccccagacatttatacggagtggaagatgaacgggcagccacaggaaaactacaagaacactccacctacgatggacacagatgggagttacttcctctacagcaagctcaatgtaaagaaagaaacatggcagcagggaaacactttcacgtgttctgtgctgcatgagggcctgcacaaccaccatactgagaagagtctctcccactctcctggtaaatgatcccagagtccagtggcccctcttggcctaaaggatgccaacacctacctctaccacctttctctgtgtaaataaagcacccagctctgccttgggaccctgcaaaaaaaaaaaaaaaaaaaaaaaaaa

SEQ ID NO: 4 - protein, 10 aa, Artificial Sequence
  Description of Artificial Sequence Synthetic peptide
EVQLVESDGG

SEQ ID NO: 5 - protein, 10 aa, Artificial Sequence
  Description of Artificial Sequence Synthetic peptide
DTVLTQSPAL

SEQ ID NO: 6 - protein, 9 aa, Artificial Sequence
  Description of Artificial Sequence Synthetic peptide
SLYNTVATL

(19) United States
(12) Patent Application Publication
(10) Pub. No.: US 2010/0291109 A1
(43) Pub. Date: Nov. 18, 2010

(54) TLR AGONIST (FLAGELLIN)/CD40 AGONIST/ANTIGEN PROTEIN AND DNA CONJUGATES AND USE THEREOF FOR INDUCING SYNERGISTIC ENHANCEMENT IN IMMUNITY

(76) Inventor: Ross Kedl, Centennial, CO (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/797,060

(22) Filed: Jun. 9, 2010

Related U.S. Application Data
(63) Continuation of application No. 11/681,092, filed on Mar. 1, 2007, now abandoned.
(60) Provisional application No. 60/777,569, filed on Mar. 1, 2006.

(51) Int. Cl. A61K 39/395; C07H 21/04; C12N 15/63; C12N 5/10; C12N 1/21; C12N 1/19; C07K 14/435; C07K 16/28; A61K 39/00; A61K 39/21; A61K 39/02; A61K 39/35; A61K 39/12; A61P 37/04; A61P 29/00; A61P 35/00; A61P 37/08; A61P 31/12; A61P 31/18; A61P 31/04; A61P 33/00; A61P 31/10; A01K 67/00

(52) U.S. Cl. 424/173.1; 536/23.53; 536/23.5; 536/23.7; 536/23.72; 536/23.74; 536/23.1; 435/320.1; 435/325; 435/349; 435/348; 435/252.3; 435/254.11; 435/366; 530/402; 530/387.3; 424/184.1; 424/208.1; 424/234.1; 424/274.1; 424/275.1; 424/277.1; 424/204.1; 800/13

(57) ABSTRACT

Fusion proteins and DNA conjugates are disclosed which contain a TLR/CD40/agonist and optional antigen combination. The use of these protein and DNA conjugates as immune adjuvants and as vaccines for treatment of various chronic diseases such as HIV infection is also provided.

### CROSS-REFERENCE TO RELATED APPLICATIONS

This application relates to and claims benefit of priority to U.S. provisional application Ser. No. 60/777,569 filed on Mar. 1, 2006. This application is incorporated by reference in its entirety herein.

### FIELD OF THE INVENTION

The invention generally relates to novel protein and DNA conjugates which promote antigen specific cellular immunity. The use of these polypeptide conjugates and DNA conjugates as immune adjuvants for treating various chronic diseases including cancer, infectious diseases, autoimmune diseases, allergic and inflammatory diseases is also taught.

### BACKGROUND OF THE INVENTION

The body's defense system against microbes as well as the body's defense against other chronic diseases such as those affecting cell proliferation is mediated by early reactions of the innate immune system and by later responses of the adaptive immune system. Innate immunity involves mechanisms that recognize structures which are for example characteristic of the microbial pathogens and that are not present on mammalian cells. Examples of such structures include bacterial liposaccharides, (LPS) viral double stranded DNA, and unmethylated CpG DNA nucleotides. The effector cells of the innate immune response system comprise neutrophils, macrophages, and natural killer cells (NK cells). In addition to innate immunity, vertebrates, including mammals, have evolved immunological defense systems that are stimulated by exposure to infectious agents and that increase in magnitude and effectiveness with each successive exposure to a particular antigen. Due to its capacity to adapt to a specific infection or antigenic insult, this immune defense mechanism has been described as adaptive immunity. There are two types of adaptive immune responses, called humoral immunity, involving antibodies produced by B lymphocytes, and cell-mediated immunity, mediated by T lymphocytes.

Two types of major T lymphocytes have been described, CD8+ cytotoxic lymphocytes (CTLs) and CD4 helper cells (Th cells). CD8+ T cells are effector cells that, via the T cell receptor (TCR), recognize foreign antigens presented by class I MHC molecules on, for instance, virally or bacterially infected cells. Upon recognition of foreign antigens, CD8+ cells undergo an activation, maturation and proliferation process. This differentiation process results in CTL clones which have the capacity of destroying the target cells displaying foreign antigens. T helper cells on the other hand are involved in both humoral and cell-mediated forms of effector immune responses. With respect to the humoral, or antibody immune response, antibodies are produced by B lymphocytes through interactions with Th cells. Specifically, extracellular antigens, such as circulating microbes, are taken up by specialized antigen-presenting cells (APCs), processed, and presented in association with class II major histocompatibility complex (MHC) molecules to CD4+ Th cells. These Th cells in turn activate B lymphocytes, resulting in antibody production. The cell-mediated, or cellular, immune response, in contrast, functions to neutralize microbes which inhabit intracellular locations, such as after successful infection of a target cell. Foreign antigens, such as for example, microbial antigens, are synthesized within infected cells and resented on the surfaces of such cells in association with Class I MHC molecules. Presentation of such epitopes leads to the above-described stimulation of CD8+ CTLs, a process which in turn is also stimulated by CD4+ Th cells. Th cells are composed of at least two distinct subpopulations, termed Th1 and Th2 cells. The Th1 and Th2 subtypes represent polarized populations of Th cells which differentiate from common precursors after exposure to antigen.

Each T helper cell subtype secretes cytokines that promote distinct immunological effects that are opposed to one another and that cross-regulate each other's expansion and function. Th1 cells secrete high amounts of cytokines such as interferon (IFN) gamma, tumor necrosis factor-alpha (TNF-alpha), interleukin-2 (IL-2), and IL-12, and low amounts of IL-4. Th1 associated cytokines promote CD8+ cytotoxic T lymphocyte T lymphocyte (CTL) activity and are most frequently associated with cell-mediated immune responses against intracellular pathogens. In contrast, Th2 cells secrete high amounts of cytokines such as IL-4, IL-13, and IL-10, but low IFN-gamma, and promote antibody responses. Th2 responses are particularly relevant for humoral responses, such as protection from anthrax and for the elimination of helminthic infections.

Whether a resulting immune response is Th1 or Th2-driven largely depends on the pathogen involved and on factors in the cellular environment, such as cytokines. Failure to activate a T helper response, or the correct T helper subset, can result not only in the inability to mount a sufficient response to combat a particular pathogen, but also in the generation of poor immunity against reinfection. Many infectious agents are intracellular pathogens in which cell-mediated responses, as exemplified by Th1 immunity, would be expected to play an important role in protection and/or therapy. Moreover, for many of these infections it has been shown that the induction of inappropriate Th2 responses negatively affects disease outcome. Examples include M tuberculosis, S. mansoni, and also counterproductive Th2-like dominated immune responses. Lepromatous leprosy also appears to feature a prevalent, but inappropriate, Th2-like response. HIV infection represents another example. There, it has been suggested that a drop in the ratio of Th1-like cells to other Th cell populations can play a critical role in the progression toward disease symptoms.

As a protective measure against infectious agents, vaccination protocols for protection from some microbes have been developed. Vaccination protocols against infectious pathogens are often hampered by poor vaccine immunogenicity, an inappropriate type of response (antibody versus cell-mediated immunity), a lack of ability to elicit long-term immunological memory, and/or failure to generate immunity against different serotypes of a given pathogen. Current vaccination strategies target the elicitation of antibodies specific for a given serotype and for many common pathogens, for example, viral serotypes or pathogens. Efforts must be made on a recurring basis to monitor which serotypes are prevalent around the world. An example of this is the annual monitoring of emerging influenza A serotypes that are anticipated to be the major infectious strains.

To support vaccination protocols, adjuvants that would support the generation of immune responses against specific infectious diseases further have been developed. For example, aluminum salts have been used as relatively safe and effective vaccine adjuvants to enhance antibody responses to certain pathogens. One of the disadvantages of such adjuvants is that they are relatively ineffective at stimulating a cell-mediated immune response and produce an immune response that is largely Th2 biased.

It is now widely recognized that the generation of protective immunity depends not only on exposure to antigen, but also the context in which the antigen is encountered. Numerous examples exist in which introduction of a novel antigen into a host in a non-inflammatory context generates immunological tolerance rather than long-term immunity whereas exposure to antigen in the presence of an inflammatory agent (adjuvant) induces immunity. (Mondino et al., Proc. Natl. Acad. Sci., USA 93:2245 (1996); Pulendran et al., J. Exp. Med. 188:2075 (1998); Jenkins et al., Immunity 1:443 (1994); and Kearney et al., Immunity 1:327 (1994)). Since it can mean the difference between tolerance and immunity, much effort has gone into discovering the “adjuvants” present within infectious agents that stimulate the molecular pathways involved in creating the appropriate immunogenic context of antigen presentation. It is now known that a good deal of the adjuvant activity is due to interactions of microbial and viral products with different members of the Toll Like Receptors (TLRs) expressed on immune cells (Beutler et al, Mol. Immunol. 40:845 (2004); Kaisho B., Biochim. Biophys. Acta, 1589 (2002):1; Akira et al., Scand. J Infect. Dis. 35:555 (2003); and Takeda K. and Akira S Semin. Immunol. 16:3 (2004)). The TLRs are named for their homology to a molecule in the Drosophila, called Toll, which functions in the development thereof and is involved in anti-microbial immunity (Lemaitre et al., Cell 86:973 (1996); and Hashimoto et al., Cell 52:269 (1988)).

Early work showed the mammalian homologues to Toll and Toll pathway molecules were critical to the ability of cells of the innate immune system to respond to microbial challenges and microbial byproducts (Medzhitov et al., Nature 388:394 (1997); Medzhitov et al., Mol. Cell 2:253 (1998); Medzhitov et al., Semin. Immunol. 10:351 (2000); Medzhitov et al., Trends Microbiol. 8:452 (2000); and Janeway et al., Annu Rev. Immunol. 20:197 (2002)). Since the identification of LPS as a TLR4 agonist (Poltorok et al., Science 282:2085 (1998)) numerous other TLR agonists have been described such as tri-acyl lipopeptides (TLR1), peptidoglycan, lipoteichoic acid and Pam3cys (TLR2), dsRNA (TLR3), flagellin (TLR5), diacyl lipopeptides such as Malp-2 (TLR6), imidazoquinolines and single stranded RNA (TLR7,8), bacterial DNA, unmethylated CpG DNA sequences, and even human genomic DNA antibody complexes (TLR9). Takeuchi et al. Int Immunol 13:933 (2001); Edwards et al., J Immunol 169:3652 (2002); Hayashi et al., Blood, 102:2660 (2003); Nagase et al., J. Immunol. 171:3977 (2003).

As noted above flagellin in particular has been previously identified as a TLR5 agonist. Based on this property the use thereof as an immune potentiator has been suggested by some groups. For example Medzhitov et al., US 20050163764 published Jul. 28, 2005 suggest the use of flagellin and other TLR agonists for treating gastrointestinal injury in a mammal by oral or mucosal administration. Also, Aderem et al., US 20050147627 published Jul. 7, 2005 teach flagellin peptides that function as TLR5 agonists and use thereof to enhance antigen-specific immune responses by co-administration of the flagellin peptide and the antigen. Further, Aderem et al. US 2003004429 published Mar. 6, 2003 teach purported flagellin peptides that function as TLR5 agonists and the use thereof to treat conditions selected from proliferative diseases (cancer) autoimmune diseases, infectious diseases and inflammatory diseases. They further disclose that this administration may be combined with an immunomodulatory molecule which may be fused thereto and may comprise an antibody, cytokine or growth factor. Still further, Dow et al., US 20050013812 published Jan. 20, 2005 teach purported vaccines comprising a toll receptor ligand and a delivery vehicle for use in treating various diseases including cancers, infectious diseases, allergic diseases, autoimmune diseases and autoimmune diseases.

The involvement of TLRs in immunity is at least 2-fold, first as direct activators of the innate immune system, such as DCs, monocytes, macrophages, NK cells, esinophils, and neutrophils (17-20) to induce a cascade of cytokines and chemokines like IFNalpha, IL-12, IL-6, IL-8, MIPlalpha and beta, and MCP-1. (Medzhitov et al., Trends Microbiol. 8:452 (2002); Kaisho et al., Cur. Mol. Med. 3:759 (2003); Kopp and Medzhitov Curr Opin. Immunol. 15:396 (2003) and Beutler et al., J Leukoc Biol. 74:479 (2003)). DCs stimulated by various TLRs become activated to increase surface expression of costimulatory markers and migrate from the tissues and marginal zones into the T cell rich area of lymphoid tissues (De Smedt et al., J Exp Med 184:1413 (1996); Doxsee et al., J Immunol 171:1156 (2003); Reis e Sousa et al., J Exp Med 186:1819 (1997); and Suzuki et al., Dermatology 114:135 (2000)). These activated DCs are ideal for the presentation of antigens, gleaned from the peripheral tissues and circulation, to CD4 and CD8+ T cells within the T cell zones. Thus, TLR stimulation induces immediate innate effector functions and also creates the necessary conditions for the initiation of adaptive immunity.

TLR agonists alone are poor adjuvants for eliciting cellular immunity. Given their ability to mediate DC activation, cytokine production, costimulatory marker expression, and migration into T cell areas of lymphoid tissue, TLR agonists would seem to be optimal for use as vaccine adjuvants. However, when compared to an actual infection, the use of purified TLR agonists as vaccine adjuvants has been disappointing at best, at least with respect to the generation of T cell responses. Within 6-9 days after infection with many viruses and bacteria, either in animal models or in the clinic, the infected host often is capable of generating pathogen-specific T cell responses constituting 20-50% of the total circulating CD8+ T cells (Busch et al., Immunol Lett 65:93 ((1999); Busch et al., J Exp Med. 189:701 (1999); Butz et al., Adv Exp Med Biol 452:111 (1998); Butz et al., Immunity 8:167 (1998)). By contrast, the generation of detectable T cell responses using only an antigen and a TLR agonist(s) often requires multiple immunizations and even then the magnitude of the T cell response is rarely better than 5-10% of the circulating CD8+ T cells (Tritel et al., J Immunol 171:2539 (2003); Will-Reece et al., J Immunol 174:7676 (2005); Rhee et al., J Exp Med 195:1565 (2002); Lore et al., J Immunol 171:4320 (2003); Ahonen et al., J Exp Med 199:775 (2004)). Thus the reduction of an infectious agent down to its antigens and TLR agonists does not reconstitute the magnitude of cellular immunity generated by the actual infection.

Another molecule known to regulate adaptive immunity is CD40. CD40 is a member of the TNF receptor superfamily and is essential for a spectrum of cell-mediated immune responses and is required for the development of T cell dependent humoral immunity (Aruffo et al., Cell 72:291 (1993); Farrington et al., Proc Natl Acad Sci., USA 91:1099 (1994); Renshaw et al., J Exp Med 180:1889 (1994)). In its natural role, CD40-ligand expressed on CD4+ T cells interacts with CD40 expressed on DCs or B cells, promoting increased activation of the APC and, concomitantly, further activation of the T cell (Liu et al Semin Immunol 9:235 (1994); Bishop et al., Cytokine Growth Factor Rev 14:297 (2003)). For DCs, CD40 ligation classically leads to a response similar to stimulation through TLRs such as activation marker upregulation and inflammatory cytokine production (Quezada et al. Annu Rev Immunol 22:307 (2004); O'Sullivan B and Thomas R Crit Rev Immunol 22:83 (2003)) Its importance in CD8 responses was demonstrated by studies showing that stimulation of APCs through CD40 rescued CD4-dependent CD8+ T cell responses in the absence of CD4 cells (Lefrancois et al., J Immunol. 164:725 (2000); Bennett et al., Nature 393:478 (1998); Ridge et al., Nature 393:474 (1998); Schoenberger et al., Nature 393:474 (1998)). This finding sparked much speculation that CD40 agonists alone could potentially rescue failing CD8+ T cell responses in some disease settings.

Other studies, however, have demonstrated that CD40 stimulation alone insufficiently promotes long-term immunity. In some model systems, anti-CD40 treatment alone insufficiently promoted long-term immunity. Additionally, in some model systems, anti-CD40 treatment alone can result in ineffective inflammatory cytokine production, the deletion of antigen-specific T cells (Mauri et al. Nat Med 6:673 (2001); Kedl et al. Proc Natl Acad Sci., USA 98:10811 (2001)) and termination of B cell responses (Erickson et al., J Clin Invest 109:613 (2002)). Also, soluble trimerized CD40 ligand has been used n the clinic as an agonist for the CD40 pathway and what little has been reported is consistent with the conclusion that stimulation of CD40 alone fails to reconstitute all necessary signals for long term CD8+ T cell immunity (Vonderheide et al., J Clin Oncol 19:3280 (2001)).

Because of the activity of TLRs and CD40 in innate and adaptive immune responses, both of these molecules have been explored as targets for vaccine adjuvants. Recently, it was demonstrated that immunization with antigen in combination with some TLR agonists and anti-CD40 treatment (combined TLR/CD40 agonist immunization) induces potent CD8+ T cell expansion, elicting a response 10-20 fold higher than immunization with either agonist alone (Ahonen et al., J Exp Med 199:775 (2004)). This was the first demonstration that potent CD8+ T cell responses can be generated in the absence of infection with a viral or microbial agent. Antigen specific CD8+ T cells elicited by combined TLR/CD40 agonist immunization demonstrate lytic function, gamma interferon production, and enhanced secondary responses to antigenic challenge. Synergistic activity with anti-CD40 in the induction of CD8+ T cell expansion has been shown with agonists of TLR1/6, 2/6, 3, 4, 5, 7 and 9. This suggests that combined TLR/CD40 agonist immunization can reconstitute all of the signals required to elicit profound acquired cell-mediated immunity.

To increase the effectiveness of an adaptive immune response, such as in a vaccination protocol or during a microbial infection, it is therefore important to develop novel, more effective, vaccine adjuvants. The present invention satisfies this need and provides other advantages as well.

### SUMMARY OF THE INVENTION

This invention provides nucleic acid constructs that encode (i) at least one TLR polypeptide, (ii) at least CD40 agonist, and (iii) optionally an antigen and the corresponding polypeptide conjugates which nucleic acid constructs or the polypeptide conjugate expressed thereby, when administered to a host in need thereof, elicit a synergistic effect on immunity, e.g., cellular immunity and more specifically primary and memory CD8+ T cell responses. By a “synergistic” effect on immunity it is intended that the DNA construct or polypeptide conjugate encoded thereby has a greater effect on immunity relative to when either of the respective agonistic polypeptides contained therein are administered alone. Particularly, this invention provides nucleic acid constructs containing a gene or genes encoding an agonistic anti-CD40 antibody, preferably an antibody against human CD40 or a soluble CD40L polypeptide or fragment or mutant thereof, and a gene encoding a polypeptide TLR agonist, preferably a TLR5 agonist (flagellin) and optionally a gene encoding an antigen against which an enhanced cellular immune response is desirably elicited.

As described in detail infra, these nucleic acid constructs or the agonist polypeptide conjugates encoded thereby may be administered to a host in need of such treatment as a means of:

(i) generating enhanced (exponentially better) primary and memory CD8+ T cell responses relative to immunization with either agonist alone;

(ii) inducing the exponential expansion of antigen-specific CD8+ T cells, and

(iii) generating protective immunity even in CD4 deficient or depleted hosts.

These nucleic acid constructs or the polypeptide conjugates expressed thereby may be used in treating any disease or condition wherein the above-identified enhanced cellular immune responses are therapeutically desirable, especially infectious diseases, proliferative disorders such as cancer, allergy, autoimmune disorders, inflammatory disorders, and other chronic diseases wherein enhanced cellular immunity is a desired therapeutic outcome. Preferred applications of the invention include especially the treatment of infectious disorders such as HIV infection and cancer. and conditions wherein subjects are CD4 deficient or depleted as a result of disease or genetic defect.

As described in detail infra such DNA constructs may comprise linear DNA, a plasmid or a viral vector containing same such as an adenoviral or baculovirus construct or other virus commonly used for gene therapy. Additionally as described infra these DNA constructs or polypeptide conjugates may be combined with other therapeutics such as other immune agonist molecules including other TLR agonists such as agonists of TLR 1, TLR2, TLR3, TLR4, TLR5, TLR6, TLR7, TLR8, TLR9, TLR10 and TLR11, or other TNFR superfamily member agonists. Examples thereof include by way of example agonists of OX40, OX40 ligand, 4-1-BB, 4-1BB ligand, CD27, CD30, CD30 ligand, HVEM, TROY, RELT, TNF-alpha, TNF-beta, CD70, RANK ligand, LT-alpha, LT-beta, GITR ligand and LIGHT. Examples of TLR agonists include MALP-2, LPS, polyIC, CpG, IRM compounds and other TLR agonists known in the art. The addition of other agonists may result in further potentiation of the immune response.

Various other features and advantages of the present invention should become readily apparent with reference to the following description, definitions, examples, claims and figures appended hereto. In several places throughout the specification guidance is provided through lists of examples. In each instance, the recited list serves only as a representative group and should not be interpreted as an exclusive list.

### DETAILED DESCRIPTION OF THE FIGURES

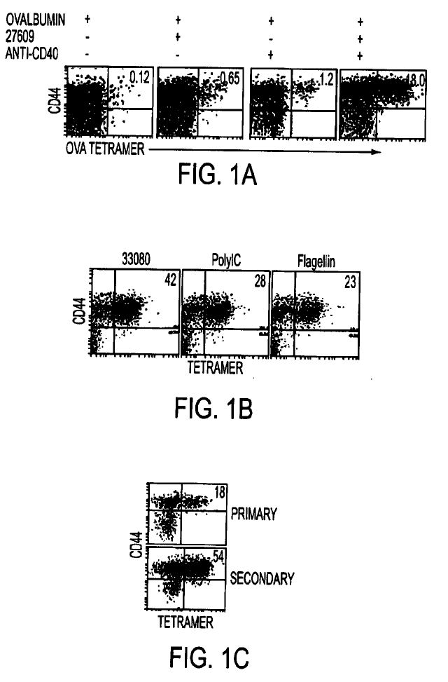

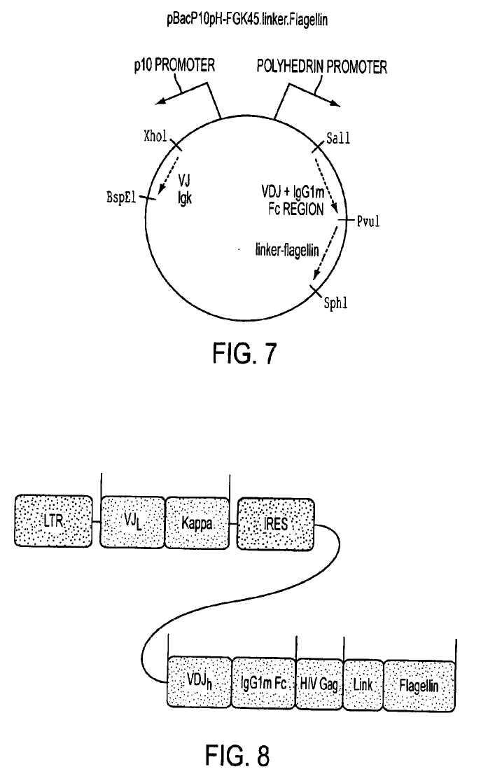

FIG. 1A-C. Panel 1A contains the results of an experiment wherein mice were immunized with combinations of antigen (ovalbumin), anti-CD40 antibody, and a TLR agonist referred to as 27609 as indicated in the Figure. 7 days later, spleen cells were removed and stained with tetramer to identify antigen-specific T cells. The data shown was gated on all CD8+ events. Numbers in the right quadrant indicate the percent of tetramer staining cells out of the total CD8 cells. Panel 1B contains the results of an experiment wherein mice were immunized with antigen (ovalbumin), anti-CD40, and the indicated TLR agonists (33080 (proprietary TLR agonist), polyIC and flagellin) and T cell responses analyzed as in Panel 1A. Panel 1C contains the results of an experiment wherein mice were immunized as in Panel B with polyIC and boosted one month later. 5 days after boosting, the T cell response in the blood was determined as in Panel 1A.

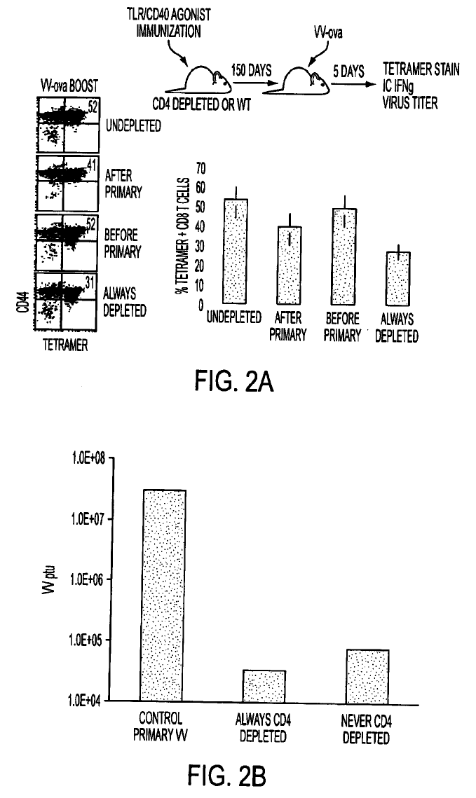

FIG. 2A-B. Panel A contains the results of an experiment wherein mice depleted of CD4 cells as described infra were immunized with ovalbumin, polyIC, anti-CD40 antibody. 150 days later the mice were challenged with 1×10×7 pfu of Vvova. 5 days after challenge, the peripheral blood was analyzed for the expansion of memory CD8+ T cells by tetramer staining as described in FIG. 1. Panel 2B contains the results of an experiment wherein the spleen and ovaries of the Vvova challenged mice in the experiment in FIG. 1A were removed and plaque assays were performed to determine viral titers.

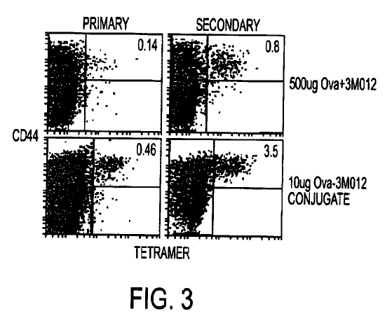

FIG. 3 contains the results of an experiment wherein mice were immunized with either 500 micrograms of ovalbumin mixed with a TLR7 agonist (3M012) or with 10 micrograms ovalbumin conjugated to the TLR7 agonist 3M012 (primary). 30 days later mice were boosted with the same (secondary). 7 days after the primary immunization and 5 days after the secondary, the antigen-specific CD8+ T cell response was determined in the blood by tetramer staining as in FIG. 1A-C.

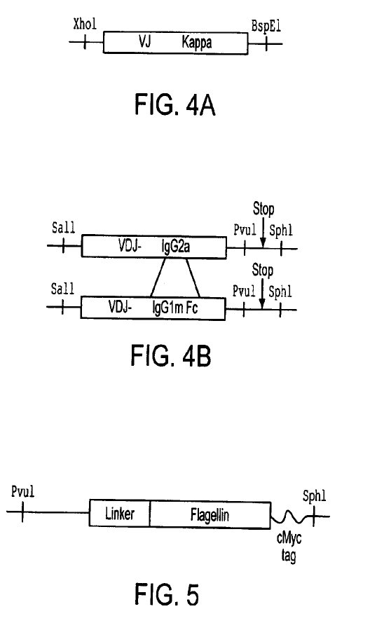

FIG. 4A-B contains a schematic of an IgG2a anti-CD40 antibody DNA construct cloned by PCR. Panel 4A depicts the cloned antibody light chain and Panel 4B depicts the cloned antibody heavy chain along with the substitution of the IgG2a constant region with an IgG1m Fc.

FIG. 5 contains a schematic of a flagellin gene containing DNA construct cloned by PCR.

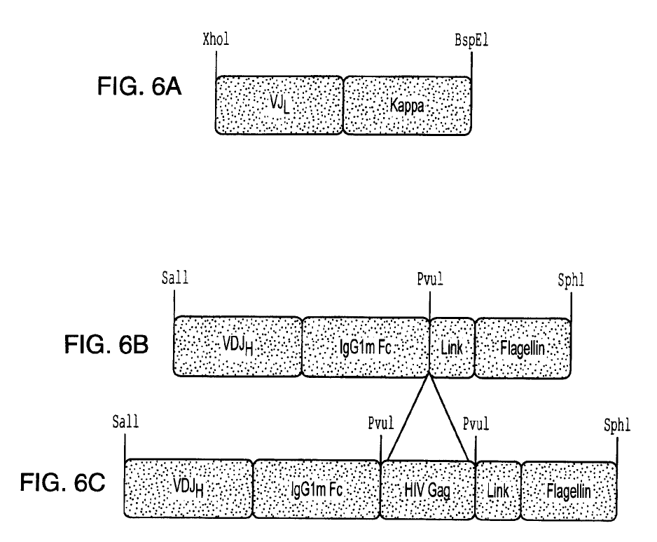

FIG. 6A-C contains a schematic of a viral antigen gene (HIV Gag sequence) integrated 3′ of the CD40 antibody heavy chain. Panel 6A shows that the Ig light chain of the cloned anti-CD40 antibody is cloned into the p10 promoter. Panel 6B shows the Ig heavy chain of the anti-CD40 antibody cloned into the construct along with a Pvu1 site for introducing a desired optional antigen gene upstream of the linker and sequence encoding flagellin (after antigen gene insertion the linker intervenes the antigen gene and the flagellin gene). Panel 6C depicts the final construct that results in the co-expression of both antibody chains and the production of a protein conjugate containing the CD40 antibody linked to a desired optional antigen, optionally a linker, and a flagellin polypeptide.

FIG. 7 depicts schematically a baculovirus expression vector construct according to the invention encoding anti-CD40 antibody light and heavy chains, and flagellin for the expression of an anti-CD40 antibody-antigen flagellin polypeptide conjugate in insect cells.

FIG. 8 depicts schematically a DNA construct according to the invention containing anti-CD40 antibody heavy and light chains, antigen (HIV Gag depicted), linker and flagellin gene.

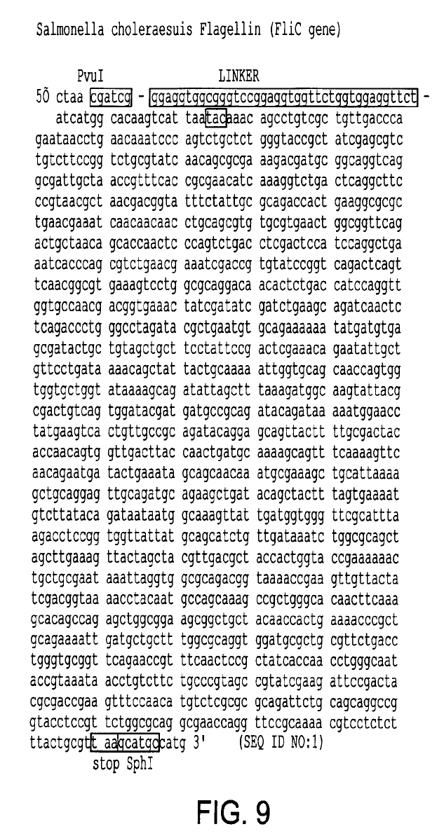

FIG. 9 contains the DNA sequence of a flagellin gene (Flic) cloned from Salmonella choleraesuis (accession number AF 159459 from NCBI nucleotide database).

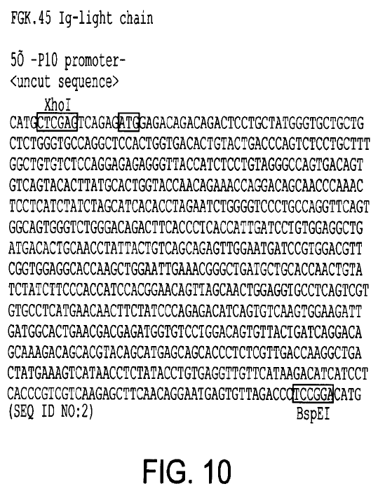

FIG. 10 contains the DNA sequence of the light chain of the anti-CD40 antibody (FGK45) used in the examples.

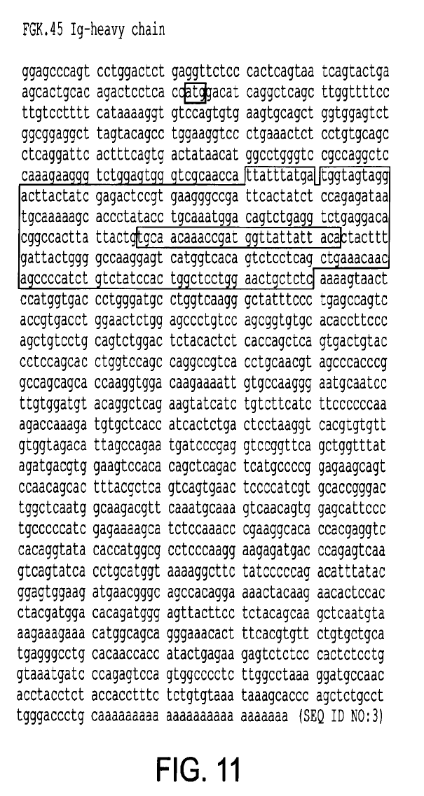

FIG. 11 contains the DNA sequence of the heavy chain of the anti-CD40 antibody (FGK45) used in the examples.

### DETAILED DESCRIPTION OF THE INVENTION

The present invention provides DNA constructs encoding a novel synergistic agonistic polypeptide combination comprising (i) a DNA encoding a specific TLR agonistic polypeptide, preferably a TLR5 agonist (flagellin) and (ii) a DNA or DNA combination encoding a specific CD40 agonist (for example a CD40L, fragment, or mutant or conjugate thereof or an agonistic antibody that binds CD40 preferably human CD40) which construct preferably optionally also includes (iii) a DNA encoding a desired antigen. These DNA constructs, vectors containing or the expression product of these DNA constructs, when administered to a host, preferably a human, may be used to generate enhanced immune responses, preferably enhanced antigen specific cellular immune responses.

The present invention further provides expression vectors and host cells containing a DNA construct encoding said novel synergistic agonistic polypeptide combination comprising (i) a DNA encoding a specific TLR agonist, preferably a TLR5 agonist (flagellin), (ii) a DNA or DNAs encoding a CD40 agonist such as a CD40L fragment, mutant or conjugate thereof or an agonistic antibody or antibody fragment that specifically binds CD40, preferably human CD40, and (iii) optionally a DNA that encodes an antigen against which enhanced antigen specific cellular immune response are desirably elicited.

Also, the invention provides methods of using said vectors and host cells to produce a composition containing said novel synergistic TLR/CD40 Agonist/Antigen polypeptide conjugate, preferably a TLR5/CD40 agonist-antigen polypeptide conjugate.

Further the invention provides methods of administering said DNA constructs or compositions and vehicles containing to a host in which an antigen specific immune response is desirably elicited, for example a person with a chronic disease such as cancer or an infectious or allergic disorder producing said composition.

Still further the invention provides compositions comprising said novel synergistic TLR/CD40 agonist antigen polypeptide conjugates which are suitable for administration to a host in order to elicit an enhanced immune response, e.g., an enhanced antigen-specific cellular immune response.

Also, the invention provides novel methods of immunotherapy comprising the administration of said novel synergistic agonist-antigen polypeptide conjugate or a DNA encoding said polypeptide conjugate to a host in need of such treatment in order to elicit an enhanced antigen specific cellular immune response. In preferred embodiments these compositions and conjugates will be administered to a subject with or at risk of developing a cancer, an infection, particularly a chronic infectious diseases e.g., involving a virus, bacteria or parasite; or an autoimmune, inflammatory or allergic condition. In an exemplary and preferred embodiment described infra, the invention is used to elicit antigen specific cellular immune responses against HIV. HIV is a well recognized example of a disease wherein protective immunity almost certainly will require the generation of potent and long-lived cellular immune responses against the virus.

As used herein the following terms shall have the meanings set forth. Otherwise all terms shall have the meaning they would normally be accorded by a person skilled in the relevant art.

“Agonist” refers to a compound that in combination with a receptor can produce a cellular response. An agonist may be a ligand that directly binds to the receptor. Alternatively an agonist may combine with a receptor indirectly by for example (a) forming a complex with another molecule that directly binds to the receptor, or (b) otherwise resulting in the modification f another compound so that the other compound directly binds to the receptor. An agonist herein will typically refer to a CD40 agonist or a TLR agonist. In some instances the subject conjugates or DNA fusions may be administered with other agonists such as other TNF/R agonists.

“Antigen” herein refers to any substance that is capable of being the target of an immune response. An antigen may be the target for example a cell-mediated and/or humoral immune response (e.g., immune cell maturation, production of cytokines, production of antibodies, etc when contacted with immune cells. Exemplary antigens are exemplified infra and include by way of example bacterial, viral, fungal polypeptides, autoantigens, allergens, and the like.

“HIV antigen” is an antigen that elicits an HIV specific immune response. Examples thereof include e.g., the HIV env, gag, and pol antigens.

“Co-administered” refers to two or more components of a combination administered so that therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component, e.g., cytokine production, activation of certain cells, etc., persists at the treatment site until one or more additional components are administered to the treatment site. Thus, a co-administered combination can in certain circumstances include components that never exist in a single chemical mixture with each other.

“Immunostimulatory combination” refers to any combination of components that can be co-administered to provide a therapeutic and/or prophylactic immunostimulatory effect. Herein the components of the immunostimulatory combination will typically comprise a CD40 agonist, a TLR agonist (e.g. flagellin) and optionally an antigen wherein all are in a single polypeptide construct or are encoded by a single DNA construct or vector. As noted these conjugates may be administered with other agonists as well such as other TNF/R agonists or other TLR agonists or cytokines.

“Mixture” refers to any mixture, aqueous or non-aqueous solution, suspension, emulsion, gel, cream or the like that contains two or more components. The components may be for example the immunostimulatory combination comprising a DNA or polypeptide conjugate according to the invention, an adjuvant or immune carrier and an antigen if one is not contained in the conjugate.

“Synergy” and variations thereof refers to activity such as immunostimulatory activity achieved when administering a combination of active agents that is greater than the additive activity of the active agents administered individually.

“Conjugate” herein refers to a single molecule, typically a DNA fusion or polypeptide fusion that contains a plurality of agonists or genes encoding and optionally an antigen or gene encoding wherein each are directly or indirectly attached to one another, e.g., by the use of linkers, and wherein these agonists and antigen if present may be in any order relative to one another in the conjugate.

TLR refers to a toll-like receptor of any species origin, e.g., human, rodent et al. Examples thereof include TLR1, TLR2, TLR3, TLR4, TLR5, TLR6, TLR7, TLR8, TLR9, TLR 10 and TLR11.

“TLR agonist” refers to a compound that acts as an agonist of at least one TLR. As noted the subject conjugates will comprise a polypeptide TLR agonist or a DNA encoding such as flagellin or a mutant or fragment thereof.

“CD40 agonist” herein refers to a molecule that functions as a CD40 agonist signal such as a CD40L polypeptide or CD40 agonistic antibody or fragment or conjugate containing. In general ligands that bind CD40 may act as a CD40 agonist. Also, CD40 agonists according to the invention may include aptamers that bind CD40.

“CD40 agonistic antibodies” herein include by way of example those available from commercial vendors such as Mabtech (Nacka, Sweden), and those reported in the literature such as those disclosed in Ledbetter et al., Crit. Rev. Immunol., 17:427 (1997) and Osada et al., J. Immunotherapy, 25(2):176 (2002). Preferably the agonistic antibody will specifically bind human CD40. Exemplary CD40 antibody variable sequences are provided infra.

Herein “CD40L” includes any polypeptide or protein that specifically recognizes and activates the CD40 receptor and activates its biological activity. Preferably it is a human CD40L or derivative or polymer or fragment thereof. Particularly the invention embraces CD40L proteins and fragments possessing at least 75-80% identity, more preferably at least 90%-95% sequence identity or more to the native CD40L polypeptide or a fragment thereof which recognize and activate the human CD40L receptor. CD40L polypeptides and corresponding nucleic acid sequences are disclosed for example in U.S. Pat. Nos. 5,565,321; 6,087,321; 6,410,711; 7,169,389; 6,264,951; US20050158831; and US20050181994 all incorporated by reference in their entirety herein.

Type 1 interferon refers collectively to known type 1 interferons such as IFN alpha, IFN beta, IFN omega, IFN tau et al. or a mixture or combination thereof.

Vaccine” refers to a pharmaceutical composition that includes an antigen. A vaccine may include components in addition to the antigen such as adjuvants, carriers, stabilizers, agonists, cytokines, et al.

“Treatment site” refers to the site of a particular treatment. Treatments sites may be the whole organism if systemic treatment or a particular site if local treatment.

“TNF/R” refers to a member of the tumor necrosis factor superfamily or the tumor necrosis factor receptor superfamily. Examples thereof include CD40, CD40L, 4-1BB, 4-1BB ligand, CD27, CD70, CD30, CD30 ligand (CD153), OX40, OX-40L, TNF-alpha, TNF-beta, TNFR2, RANK, LT-beta, LT-alpha, HVEM. GITR, TROY, RELT, of any species and allelic variants and derivatives thereof.

The present invention is an extension of the prior demonstration by an inventor of this patent application and others that immunization with antigen in the presence of agonists for both a toll-like receptor (TLR) and CD40 (combined TLR/CD40 agonist immunization) elicits a vigorous expansion of antigen specific CD8+ T cells. The response elicited from this form of vaccination is exponentially greater than the response elicited by either agonist alone, and is far superior to vaccination by conventional methods. Combined TLR/CD40 agonist immunization produces potent primary and secondary CD8+ T cell responses, achieving 50-70% antigen specific T cells in the circulation after only 2 immunizations. However, unlike the inventors' prior invention, herein the TLR agonist, the CD40 agonist, and optionally an antigen are preferably administered as a single polypeptide fusion of these three entities or in the form of a DNA conjugate or vector or virus or other cell encoding or expressing said three separate entities. This is advantageous in the context of a polypeptide or DNA based vaccine since potentially only one active agent will need to be formulated and administered to a subject in need of treatment, for example an individual with HIV infection.

Also, the invention is an extension of studies from other researchers which have shown that while primary CD8+ T cell responses proceed normally in CD4 depleted or deficient hosts, that memory CD8 responses are diminished 5-10 fold compared to wild-type hosts. It is show herein that both primary and memory CD8+ T cell responses elicited by combined TLR/CD40 agonist administration occurs independent of CD4+ T cells. This is a feature unique of the subject invention (in comparison to the prior art) and is a necessary component for a vaccination to be useful in treating HIV infected individuals where CD4 T cell function is impaired.

It is now largely agreed that primary and memory CD8+ T cell responses display a differential dependence on the presence and/or function of CD8+ T cells. Primary CD8+ T cell responses can be easily generated in CD4 deficient hosts in response to a variety of stimuli. In general, the stimulation of CD40 alone facilitates the induction of CD4-independent primary CD8+ T cell responses (Bennett et al., Nature 393:478 (1998); Ridge et al., Nature 393:474 (1998); Schoenberger et al., Nature 393:480 (1998)) and was even shown to facilitate memory CD8+ T cell responses in some model systems (O'Sullivan B and Thomas R, Crit. Rev Immunol 22:83 (2003); Sotomayor et al., Nat Med 5:780 (1999); Diehl et al., Nat Med 5:774 (1999)). However, more recent data from a number of groups has indicated that, independent of the stimulus used to generate the primary response, memory CD8+ T cell responses appear to be critically dependent on the presence of CD4 cells (Grakoui et al., Science 302:569 (2003); Janssen et al., Nature 481:852 (2003); Janssen et al., Nature 434:88 (2005); Shedlock et al., Science 300:337 (2003); Sun et al., Science 300:339 (2003); Sun et al., Nat Immunol 5:927 (2004)). There is some discrepancy in the literature regarding whether CD4 cells are necessary during the activation or effector/memory phase of the primary response, but in either case, the elimination of CD4+ T cells at the appropriate time has profound impact on the survival and function of memory CD8+ T cells. To date there has not been identified a method of immunization that can generate CD4 independent, CD8+ T cell memory. The identification of such a method is significant given the deficiency of effective CD4 T cell responses in many devastating chronic human diseases such as AIDS.

With particular respect to attenuated microbial and viral vaccines, it is well known that some live attenuated vaccines can generate potent cellular and humoral immunity. (Cox et al., Scand J Immunol 59:1 (2004); Hobson et al., Methods 31:217 (2003); Polo et al., Drug Disc Today 7:719 (2002)61-63). However, numerous problems exist with these vaccines ranging from the practical concerns of vaccine production and storage to public health issues such as adverse reactions or reversion to virulence in some portion of the population. Additionally, not all pathogens, such as HIV and other viruses or other microbial pathogens, can be attenuated for use as a vaccine. Therefore, many practical vaccines, and ones more successful at eliciting cellular immunity are needed.

As noted previously, unfortunately, the majority of vaccine adjuvants developed to date have not demonstrated the ability to generate clinically significant cell mediated immunity. Both TLR agonists (Hadden et al., Int J Immunopharmacol 16:703 (1994); McElrath, M J Semin Cancer Biol 6:375 (1995); Thoelen et al., Vaccine 19:2400 (2001); Podda et al., Expert Rev Vaccines 2:197 (2003); Audibert F., Int Immunopharmacol 3:1187 (2003)) and a CD40 agonist (Vonderheide et al., J Clin Oncol. 29:3280 (2001); Ottaiano et al., Tumori 88:361 (2002); Murali-Krishna et al., Adv Exp. Med Biol 452:123 (1998)) have been used separately in the clinic but the magnitude of the responses generated has not yet warranted their FDA approval. Therefore, there is a significant need for the development and implementation of new vaccine adjuvants and/or adjuvant formulations that are able to generate potent T cell immunity.

The present invention satisfies this need by providing novel vaccine adjuvants that include at least one TLR polypeptide agonist, preferably a TLR5 agonist (flagellin), at least one polypeptide CD40 agonist (anti-CD40 antibody or fragment thereof or a CD40L polypeptide or fragment, mutant or conjugate thereof such as a trimeric CD40L) and preferably at least one antigen against which enhanced antigen-specific cellular immunity is desirably elicited. In the preferred embodiment of the invention these polypeptide moieties will be contained in a single polypeptide conjugate or will be encoded by a nucleic acid construct which upon expression in vitro in a host cell or in vivo upon administration of a naked DNA or host cell containing to a host results in the expression of said agonists and antigen polypeptides or the expression of a conjugate containing these polypeptides.

While it has been previously reported that TLR agonists synergize with anti-CD40 for the induction of CD8+ T cell immunity. to date all these studies have required the separate administration of the antigen, the TLR agonist and the CD40 agonist. By contrast this invention provides DNA constructs and tripartite polypeptides that comprise all three of these moieties or a DNA encoding all three of these moieties in a single DNA or polypeptide molecule. This will simplify the use thereof for vaccine purposes since only one molecular entity will need to be formulated in pharmaceutically acceptable form and administered. This is particularly advantageous in the context of the treatment of a chronic disease or condition wherein large amounts of adjuvant may be required for effective prophylactic or therapeutic immunity.

In the case of flagellin specifically, this TLR agonist was observed by the present inventors to exhibit some characteristics not shared by other tested TLR agonists when used in combination with a CD40 agonist (CD40 antibody). Particularly, while other TLR agonists (TLR3,7 agonists) which yielded good memory responses when boosted with antigen (months after immunization with the particular agonist, CD40 antibody and antigen these animals were boosted with the same antigen) that in all instances this memory response was accompanied by type 1 interferon induction. By contrast, for all other TLR agonists (other than flagellin) which did not elicit good memory responses upon antigen boosting (particularly TLR2,4) it was observed that boosting was not accompanied by the induction of type 1 interferon production. This is advantageous given the known role of type 1 interferons in CD8+ immunity. However, surprising is that the flagellin (TLR5) agonist CD40 antibody combination when administered to mice which were similarly boosted with the antigen months later did elicit a good memory response notwithstanding the fact that type 1 interferon production was not concomitantly induced. This would suggest that while TLRs share many properties that are involved in adaptive immunity that there are some differences which affect cellular immune reactions elicited thereby. Particularly, it suggests that different TLR agonists may elicit different effects on cellular immunity and that these differences may be significant in the context of specific diseases treatments. These observations with flagellin are believed to be unexpected.

The results disclosed herein with the exemplified conjugates support a conclusion that the subject agonist conjugates or DNA encoding when administered to a host in need thereof will generate and maintain protective cellular immunity in both normal and CD4 deficient hosts following immunization with a combined TLR/CD40 agonist polypeptide conjugate or DNA based vaccine according to the invention. This may be confirmed by observing for the induction of protective immunity against both systemic and mucosal viral challenge since mucosal immunity may also be significant to an effective vaccine especially against HIV, or other viruses such as herpes and HPV which transmit through the genital mucosa.

With respect thereto, while CD40 agonists and TLR agonists have been used separately in prior clinical studies, and flagellin (TLR5 agonist) and anti-CD40 antibody in particular, combining these agonists into a therapeutic or prophylactic vaccine formulation and the use especially in the treatment of chronic diseases such as cancer, infection, allergy, and autoimmune diseases is novel to this invention.

Combined TLR/CD40 agonist immunization, using only molecular reagents, uniquely generates CD8+ T cell responses of a magnitude that were previously only obtainable after challenge with an infectious agent (Ahonen et al., J Exp Med 199:775 (2004)). Our findings, shown e.g., in FIG. 1 demonstrate the success of this immunization in generating CD8+ T cell memory even in CD4 depleted hosts. This is surprising and exciting given that other immunization techniques where memory CD8+ T cell responses are critically dependent upon the presence of CD4+ T cells. The generation of CD4-independent CD8+ T cell responses provides for the development of therapies of many chronic diseases such as cancer, and infectious diseases like HIV et al. where a functional CD4+ T cell response is impossible or problematic. Thus, this invention provides for the development of potent vaccines against HIV and other chronic infectious diseases involving viruses, bacteria, fungi or parasites as well as proliferative diseases such as cancer, autoimmune diseases, allergic disorders, and inflammatory diseases where effective treatment requires the quantity and quality of cellular immunity that combined TLR/CD40 agonist immunization is capable of generating.

Exemplification of the Invention with Model Antigens

Combined TLR/CD40 Agonist Immunization Generates Primary and Memory CD8+ T Cell Responses

Immunization in the context of either TLR agonists or anti-CD40 alone is capable of initiating a CD8+ T cell response to antigenic challenge. However, antigenic challenge in the context of combined TLR/CD40 agonist immunization demonstrates a synergy for inducing the expansion of CD8+ T cells that cannot be reproduced with any tested amount of either agonist alone. (Ahonen et al. J Exp Med 199:775 (2004)) In initial experiments, mice were immunized with whole ovalbumin alone, with a proprietary TLR7 agonist compound S27609 (Doxsee et al., J Immunol. 171:1156 2003) or with anti-CD40 antibody, or both The antigen specific CD8+ T cell response generated from the combined TLR7/CD40-agonist immunization comprised anywhere from 5-20% of the total CD8+ T cells in the spleen (see FIG. 1A) and 15-40% of the CD8+ T cells in the blood.

To determine whether this synergistic activity was specific to TLR7 agonists or was a property of TLR agonists in general, mice were challenged with the indicated combinations of whole ovalbumin, anti-CD40 and a number of other TLR agonists. These included poly IC (TLR3), flagellin (TLR5) and a proprietary TLR agonist compound 33080 (TLR7 agonist). (See FIG. 1B) All TLR agonists were able to synergize with anti-CD40 to induce varying levels of CD8+ T cell expansion depending on the TLR agonist used. CD8+ T cell responses generated by combined TLR/CD40 agonist administration were found to be functional with respect to lytic activity, gamma interferon production (Ahonen et al. (Id)) and the ability to mount a memory response to secondary antigenic challenge. Mice previously immunized in the context of combined TLR/CD40 agonists were re-challenged 1 month later with the same immunization. The secondary expansion of the ovalbumin specific T cells was determined by tetramer staining of cells in the peripheral blood isolated 5 days after re-challenge. The peak of a primary response in the blood is between days 6 and 8 so the detection of tetramer staining cells on day 5 is an indication that they are derived from a secondary response (see FIG. 1C). It is noted that the secondary response generated by this immunization is similar in magnitude to the secondary response to an infectious agent such as LCMV. Thus, combined TLR/CD40 agonist administration not only generates potent primary CD8+ T cell immunity but also generates potent memory CD8+ T cell responses as well. No other molecular based vaccine either preclinical or clinical has been publicly disclosed that is capable of generating this magnitude of CD8+ T cell response after only 2 immunizations.

Combined TLR/CD40 Agonist Immunization Generates CD8+ T Cell Memory in CD4 Depleted Hosts

As afore-mentioned, numerous recent reports have demonstrated that a functional memory CD8+ T cell response is dependent upon the presence of CD4+ T cells (Grakoui et al., Science 302:569 (2003); Janssen et al., Nature 421:852 (2003); Janssen et al., Nature 434:88 (2005); Shedlock et al., Science 300:337 (2003); Sun et al., Science 300:339 (2003); and Sun et al., Nat Immunol 5:927 (2004)) The exact stage of the CD8+ T cell response that is dependent upon the presence of CD4 T cells is somewhat under dispute (Grakoui et al., Science 302:659 (2003); Sun et al., Nat Immunol. 5:927 (2004)) but collective data generally supports the conclusion that long term CD8+ T cell memory responses are CD4 dependent. Therefore, the fate of CD8+ T cells elicited by combined TLR/CD40 agonist immunization in CD4 depleted hosts was examined.

In order to determine what, if any stage of the CD8+ T cell response was dependent on CD4 cells, half of the non-depleted mice were treated with anti-CD4 antibody on day 6 at the same time half of the depleted mice were stopped being given anti-CD4 (See FIG. 5) This resulted in four groups of mice; ten never CD4 depleted; twenty CD4 depleted only once before the primary response; thirty CD4 depleted only after the primary response and forty always CD4 depleted. One hundred and fifty days after initial priming with combined TLR/CD40 agonist immunization, the inventors challenged the mice with a vaccinia virus expressing ovalbumin (Vvova) (Kedl et al., Proc Natl Acad Sci, USA 98:10811 (2001); Kedl et al., J Exp Med 192:1105 (2000)). Five days after Vvova challenge, the expansion of the ovalbumin specific CD8+ t cells in the peripheral blood by tetramer staining as described above was examined (See FIG. 6) As shown therein, it was observed that all mice, including CD4 depleted, generated a robust secondary T cell response.

Mice CD4 depleted either before or after the primary immunization demonstrated essentially no difference in their secondary CD8+ T cell response to Vvova. The only difference seen was an approximate 2-fold reduction in the percentage of CD8+ T cells at the peak of their secondary response in the mice always depleted of CD4 cells compared to the mice in any other group (FIG. 2A). While this is a reduction, a secondary response of this magnitude (30-40% antigen specific T cells) appears far from hyporesponsive. This is in contrast to previous reports that have demonstrated 10 fold or greater reduction in CD8 memory responses from CD4 depleted hosts compared to non-depleted hosts. In addition, virus titers in both CD4 depleted and non-depleted mice were essentially identical (FIG. 2B) indicating that the 2-fold reduction in T cell numbers did not have a corresponding impact on the degree of protective immunity conferred. Almost identical results were obtained in class II knockout (CII KO) mice which are CD4 deficient (data not shown). The data collectively demonstrate that combined TLR/CD40 agonist immunization successfully reconstitutes the majority of the signals necessary to promote CD8+ T cell mediated protective immunity even in the absence of CD4+ T cells.

Covalent Linkage of a TLR7/TLR8 Agonist to Antigen Enhances the Generation of CD8+ T Cells

Recently a small molecule TLR7/8 agonist covalently linked to an antigen was reported to enhance the production of both CD4+ and CD8+ T cell immunity. (Wille Reece et al., J Immunol. 174:7676 (2005)) These small molecules, generally fall in a family of molecules known as imidazoquinolines, have been modified with a UV-activated crosslinker and as such can be easily attached to a protein of interest such as an antigen or antibody. In preliminary experiments, the antigen-TLR7/8 agonist conjugate generated detectable CD8+ T cell responses at 50-10 fold lower antigen doses than did immunization with unconjugated antigen mixed with the TLR7/8 agonist (See FIG. 3). These results demonstrate the potential feasibility of covalently attaching an immunologically active agonist against TLR7/8 to a binding protein.

The results of the previous experiments with a model antigen ovalbumin demonstrate that combined TLR/CD40 agonist immunization is exponentially better at generating primary and memory CD8+ T cell responses than immunization with either agonist alone; that all known TLR agonists synergize with anti-CD40 to induce this exponential expansion of antigen specific CD8+ T cells; and that this means of immunization is able to generate protective immunity even in CD4 deficient or depleted hosts. The demonstration that flagellin, a TLR5 ligand/agonist, effectively synergizes with anti-CD40 for inducing CD8+ T cell expansion is important because, unlike all other TLR agonists, it is completely protein based and as such can be expressed in recombinant form. Based thereon, the inventors conceived the production of a DNA vector encoding for the expression of a covalently linked form of all constituents in the combined TLR/CD40 agonist vaccine; i.e., an antigen, anti-CD40 and a TLR agonist (flagellin). Thereby, the inventors were able to determine whether this conjugate can be used a single entity molecule based recombinant vaccine.

Based on these results, the inventors have constructed DNA vectors that should in the context of HIV immunization generate a potent cellular immune response against HIV by producing a recombinant polypeptide comprising the HIV Gag protein as the antigen, flagellin as the TLR agonist and an anti-CD40 antibody as the CD40 agonist. This conjugate should generate potent HIV Gag-specific protective cellular immunity in a systemic and mucosal viral challenge model. For the reasons set forth previously, HIV Gag was selected since HIV is an important example of a disease wherein the efficacy of a protective or therapeutic vaccine will likely require that such vaccine generate an enhanced and prolonged cellular immune response in an immunized host. However, as afore mentioned, this, invention broadly encompasses the use of the subject immune adjuvant polypeptide conjugates and DNA constructs encoding such polypeptide conjugates to elicit enhanced cellular immune responses against any desired antigen, preferably one that correlates to and/or is expressed in a chronic disease such as cancer, autoimmune disorder, allergy, inflammatory or infectious disease.

### Exemplification of Invention for Producing Infectious Disease Vaccine (HIV Vaccine) Using HIV Gag Antigen

Methods and Materials

The production of the conjugate for producing the subject therapeutic vaccine requires obtaining e.g. by cloning of DNAs encoding an anti-CD40 antibody, flagellin, and HIV Gag antigen and inserting said sequences into a vector such that they are transcribed under the control of a regulatory sequence that provides for the expression of a polypeptide conjugate containing all of these entities. Particularly, as exemplified herein a vector was constructed containing DNA sequences encoding the heavy and light chains of an IgG2a anti-CD40 antibody (wherein the IgG2a constant region is substituted with IgG1m constant region), and further wherein said light and heavy chain DNA sequences are separated by an IRES, the heavy Ig chain is linked to the HIV Gag antigen gene, and wherein such antigen gene is joined to a DNA encoding a flagellin with an intervening linker encoding a linker polypeptide of 15 amino acids. Thus in the resultant conjugate the antigen is attached to the carboxy end of the heavy chain of the anti-CD40 antibody and the flagellin is in turn attached to the antigen by means of a linker polypeptide. However, while this is exemplified it is alternatively possible to attach the antigen and the flagellin directly or indirectly to the antibody light chain in the DNA construct. Also, the antigen gene and the flagellin gene may optionally be intervened by an IRES and/or the antibody light chain sequences and the antigen gene may further optionally be intervened by an IRES. Also, the antibody may be a single chain antibody (scFv) or an antibody fragment rather than an intact multichain antibody.

Cloning of Anti-CD40, Flagellin and HIV Gag Sequences

The cloned anti-CD40 antibody sequences are that of an IgG2a monoclonal antibody which is secreted by the FGK45 hybridoma. The flagellin gene is obtained from genomic DNA cloned from S minnesota. The HIV Gag gene is obtained from a recombinant strain of vaccinia virus that expresses the entire Gag protein (kindly provided by Robert Seder, NIH Vaccine Research Center). As noted previously these sequences can be assembled in the vector in various combinations. Also, other sequence may be included such as selectable markers, affinity tags, and the like.

Cloning of Antibody Genes

The FGK45 hybridoma makes an IgG2a anti-CD40 antibody. The purified antibody was run on a reducing gel, the heavy and light chains bands cut out from the gel, and N-terminal sequencing was effected for both chains. The sequence derived from heavy chain analysis was determined to be EVQLVESDGG which corresponds to the Vh3 region. The light chain N terminal sequence was determined to be DTVLTQSPAL and was determined to correspond to the kappa light chain sequence. 3′ primers were synthesized based on the database sequence for IgG2a and kappa. Degenerate 5′ primers were made based on the amino acid sequence data from N-terminal sequencing. For both the 5′ and 3′ primers, Xho1, BspE1, Sal1, Pvu1, and Sph1 cut sites (FIG. 4) were incorporated in order to generate the necessary PCR products for cloning. The Pvu1 site is used to clone in sequences encoding the target HIV Gag antigen sequence and sequences encoding other desired antigens. A stop codon has been incorporated into the construct such that recombinant antibody-antigen protein can be produced without incorporating flagellin.

The flic gene encodes the portion of flagellin that is active in stimulating TLR5. Based on the database sequence information, primers were constructed to facilitate the cloning of flagellin from the S minnesota bacterium genome. (FIG. 5) The primers incorporate a 5′ Pvu1 cut site for ligation downstream of the heavy chain insert shown in the figure and a 3′ Sph1 cut site for ligation into the vector. Downstream of the Pvu1 cut site, the 5′ primer also encodes for a 15 amino acid linker consisting of 5 repeats of the sequence (GYS). The purpose of this linker is to provide greater distance from the heavy chain and the antigen and thereby facilitate interaction of the resultant protein conjugate with both TLR5 and CD40 on the targeted dendritic cell surface. Upstream of the Sph1 cut site, the 3′ primer also encodes for a cMyc epitope tag for the purpose of eventual affinity purification of the recombinant protein product.

Primers modified to encode Pvu1 cut sites on both the 5′ and the 3′ ends are used to generate a p41 Gag PCR product from pP41hxb2 plasmid (FIG. 6). While the HIV Gag sequence is the model antigen initially, for immunization studies, a DNA a sequence encoding ovalbumin, and the vaccinia virus B8R epitope (Tscharke et al., J Exp Med 201:95 (2005)) are cloned as these antigens are used as controls.

Using these primers, the respective PCR products are cloned into two separate vectors for making protein or DNA based vaccines. For protein production, the baculovirus bi-cistronic vector pBacp10Ph vector is used. This vector has two promoters, the polyhedron and p10 (FIG. 7). The Ig light chain is cloned into the Xho1 and BspE1 cloning sites downstream of the p10 promoter. The heavy chain is cloned into the Sal1 and Sph1 sites downstream of the polyhedron promoter. The PCR primer used to clone the heavy chain encodes both a Pvu1 and Sph1 cut sites with an intervening stop codon. Following cloning of the heavy chain, the Fc region of the IgG2a is replaced with an IgG1 Fc that has been mutated to prevent binding to Fc receptors. (Clynes et al., Nat Med 6:443 (2000)) (Kindly provided by Jeff Ravetch and Michael Nussenzweig, Rockefeller University).

The Pvu1 site is maintained and used for cloning the sequence encoding the flagellin-linker. Additionally, the Pvu1 site is used for cloning the HIV Gag sequence and for incorporating other antigen genes into the construct. The final product encodes the Ig light chain of the anti-CD40 antibody under the control of the p10 promoter and the heavy chain-IgG1mFc-HIVGag-linker-flagellin expressed under the control of the polyhedron promoter. (FIG. 7)

When the subject sequences are used in a DNA based vaccine (naked DNA or DNA incorporated into a suitable vehicle such as a virus or a liposomal delivery system) the PCR products are preferably cloned into the pVS53 expression vector. This vector drives protein expression by means of CMV LTRs and it has been previously used by the inventors for DNA-based immunizations (unpublished results). The kappa light chain PCR product is placed 5′ proximal to the CMV LTR promoter followed by an internal ribosomal entry site (RES) cloned from the pUBI-GFP vector. The heavy chain VDJ, mutant IgG1 Fc portion, HIV Gag, and flagellin are cloned following the IRES as indicated in FIG. 8.

TLR7/8 Conjugates to Anti-CD40-HIV Gag-Flagellin

Numerous DC subsets exist in both mouse and man, each expressing both common and unique TLRs. Significantly more is known about mouse DCs where the direct ex vivo analysis of DC subsets derived from different lymphoid and peripheral tissues is possible. By contrast, less is known concerning DC subsets other than those that can be identified in the blood or differentiated from monocytic precursors isolated from blood. It is therefore unclear which DC subsets are necessary to engage in antigen presentation in order to effectively generate a T cell response. Additionally, it is unclear which TLRs are necessary to target in order to achieve full activation of the appropriate DC subset. Effective vaccination may require the ability to target either multiple TLRs on a given DC and/or multiple DD subsets expressing different TLRs. To that end, the invention further embraces a vaccine consisting of antigen and anti-CD40 antibody coupled to other polypeptide TLR agonists, e.g., an agonist for TLR7/8 as described above.

Vector Construction

Baculovirus is made from the constructs shown in FIG. 7 and digested baculovirus plasmid DNA as previously described. (Rees et al., Proc Natl Acad Sci., USA 96:9781 (1999)) Following virus production and cloning, Hi5 cells are infected and 5-7 days later the supernatant harvested, filtered, and the recombinant, TLR5/CD40-agonist conjugate protein purified using an anti-Myc affinity column. The amount of protein is quantified and tested for activity against CD40 and TLR5. TLR5 activity is verified using a TLR5HEK293 transfectant and NfkappaB reporter assay system as previously described (Doxsee et al., J Immunol 171:1156 (2003); Gibson et al., Cellular Immunology 218:74 (2002)). Anti-CD40 activity is verified based on B cell and DC activation in MyD88 knock out (KO) mice as previously described (Doxsee et al. (Id)). MyD88 KO mice are deficient in signaling through most TLRs, including TLR5. As such any activation of DCs or B cells observed in these mice following injection of the recombinant protein vaccine must be due to the activity of the anti-CD40 antibody.

Two forms of the recombinant protein are made; the light chain, heavy chain and HIV Gag with and without flagellin. The TLR7/8 agonist is conjugated to each other resulting in the production of proteins which stimulate CD40/TLR7/8. A proprietary TLR7/8 agonist, called 3M012 (3M Pharmaceuticals Inc, St. Paul Minn.) contains a photoconjugatable linker, which when placed under UV light, conjugates rapidly to terminal amino groups (lysines, arginines, N terminus) in the protein of interest. Conjugation to the TLR7/8 agonist is performed as described previously (Wille-Reece et al., J Immunol. 174:7676 (2005)). Briefly, recombinant protein is placed in deep well polypropylene 96 well plates (Costar) with 50-100 microliters of 10 mg/ml 3M012 and exposed to longwave UV light for 15 minutes. Following UV exposure, the recombinant protein-TLR7/8 conjugate is washed through a 30 kd cutoff centricon concentrator to remove any free 3M012 and higher molecular weight drug conjugates. The recombinant protein-TLR7/8 conjugates are washed in PBS pH 7.5-8 and analyzed as described above for anti-CD40 and TLR5 activity (for recombinant protein containing flagellin). The amount of 3M012 conjugated to the recombinant protein is determined in vitro by type 1 IFN (IFNalphabeta) induction in spleen cells as previously described (Wille-Reece et al. (Id)). Flagellin does not induce IFN alphabeta so recombinant proteins that contain flagellin will not aberrantly influence the calculation of 3M012 conjugation. While TLR7/8 activity can be determined by induction of luciferase activity in a TLR7/8 transfectant HEK293 NfkappaB expression system (Doxsee et al. (Id)., Gibson et al., (Id)) it has been found that this is not as sensitive an assay as IFNalphabeta induction from normal spleen DCs (unpublished results).

The primers described above have resulted in the successful cloning of the anti-CD40 heavy and light chain DNA sequences as well as the flagellin Flic gene from S minnesota. The anti-CD40 antibody light and heavy chain sequences are contained in FIG. 10 and FIG. 11 respectively. The sequence for the cloned flagellin DNA is contained in FIG. 9. These sequences have been cloned into the pBacp10 vector.

The exemplified vector depicted in FIG. 7 provides for the co-expression of both Ig chains resulting in an anti-CD40 antibody linked to a desired antigen (HIV Gag) which is attached to the antigen via a linker. Thus the expression product which elicits a synergistic effect on antigen specific cellular immunity upon administration is a discrete molecular entity that contains the antigen, flagellin (TLR5 agonist) and the anti-CD40 antibody (CD40 agonist)

A baculoviral construct was selected because it is well known for producing protein antigens and MHC class I and Class II tetramers. Also, this expression system provides for high protein yields. This is desirable given the intended in vivo applications of the subject recombinant protein. It is likely that the subject conjugate when used as a therapeutic for treating a chronic disease condition which will require large amounts of protein.

Because the expression system may result in aberrant glycosylation the function of the recombinant agonists is confirmed using the afore-described assays. If determined to be problematic, this may be avoided by alternatively expressing the conjugate in a mammalian expression system. Alternatively, the glycosylation sites may be removed by mutagenesis of the flagellin and/or antibody sequences thereby precluding insect cell glycosylation. Particularly if insect cell glycosylation is problematic the DNA may be cloned into a Cos or CHO expression vector system.

### APPLICATIONS OF THE INVENTION

The invention relates to DNA conjugates or the corresponding polypeptide conjugates or polypeptides expressed thereby containing a CD40 agonist, a TLR polypeptide agonist such as flagellin or another polypeptide TLR agonist, and optionally an antigen and the use thereof alone or in association with other agonists or cytokines in promoting cellular immune responses. In particular the inventors exemplify herein both protein and DNA based vaccines comprising (i) anti-CD40-HIV Gag-flagellin; and (ii) anti-CD40-HIV Gag-flagellin. As mentioned, HIVGag40 was selected as a model antigen because HIV is a chronic infectious disease wherein an enhanced cellular immune response has significant therapeutic potential. However, the invention embraces the construction of conjugates as described containing any antigen against which an enhanced cellular immune response is therapeutically desirable. In the preferred embodiment the antigen is comprised in the administered polypeptide conjugate or is encoded by the administered DNA. However, in some embodiments a conjugate containing flagellin and the anti-CD40 antibody may be administered separate from the antigen, or the host may be naturally exposed to the antigen. Additionally, in some embodiments all three moieties, i.e., the anti-CD40 antibody or other CD40 agonist such as a CD40L or conjugate or fragment thereof; the flagellin or other TLR5 agonist polypeptide; and an antigen may be co-administered as separate discrete entities. Preferably all these moieties are administered substantially concurrently in order to achieve the desired synergistic enhancement in cellular immunity. These moieties may be administered in any order.

Exemplary antigens for use in the present invention include but are not limited to bacterial, viral, parasitic, allergens, autoantigens and tumor associated antigens. If a DNA based vaccine is used the antigen will be encoded by a sequence contained in the administered DNA construct. Alternatively, if the antigen is administered as a conjugate the antigen will be a protein comprised in the administered conjugate. Still further, if the antigen is administered separately from the CD40 antibody and the flagellin moieties the antigen can take any form. Particularly, the antigen can include protein antigens, peptides, whole inactivated organisms such as viruses, bacteria, fungi, and the like.

Specific examples of antigens that can be used in the invention include antigens from hepatits A, B, C or D, influenza virus, Listeria, Clostridium botulinum, tuberculosis, tularemia, Variola major (smallpox), viral hemorrhagic fevers, Yersinia pestis (plague), HIV, herpes, pappilloma virus, and other antigens associated with infectious agents. Other antigens include antigens associated with a tumor cell, antigens associated with autoimmune conditions, allergy and asthma. Administration of such an antigen in conjunction with the subject agonist combination flagellin and an anti-CD40 antibody or CD40L type CD40 agonist can be used in a therapeutic or prophylactic vaccine for conferring immunity against such disease conditions.

In some embodiments the methods and compositions can be used to treat an individual at risk of having an infection or has an infection by including an antigen from the infectious agent. An infection refers to a disease or condition attributable to the presence in the host of a foreign organism or an agent which reproduce within the host. A subject at risk of having an infection is a subject that is predisposed to develop an infection. Such an individual can include for example a subject with a known or suspected exposure to an infectious organism or agent. A subject at risk of having an infection can also include a subject with a condition associated with impaired ability to mount an immune response to an infectious agent or organism, for example a subject with a congenital or acquired immunodeficiency, a subject undergoing radiation or chemotherapy, a subject with a burn injury, a subject with a traumatic injury, a subject undergoing surgery, or other invasive medical or dental procedure, or similarly immunocompromised individual.

Infections which may be treated or prevented with the vaccine compositions of this invention include bacterial, viral, fungal, and parasitic. Other less common types of infection also include are rickettsiae, mycoplasms, and agents causing scrapie, bovine spongiform encephalopathy (BSE), and prion diseases (for example kuru and Creutzfeldt-Jacob disease). Examples of bacteria, viruses, fungi, and parasites that infect humans are well know. An infection may be acute, subacute, chronic or latent and it may be localized or systemic. Furthermore, the infection can be predominantly intracellular or extracellular during at least one phase of the infectious organism's agent's life cycle in the host.

Bacterial infections against which the subject vaccines and methods may be used include both Gram negative and Gram positive bacteria. Examples of Gram positive bacteria include but are not limited to Pasteurella species, Staphylococci species, and Streptococci species. Examples of Gram negative bacteria include but are not limited to Escherichia coli, Pseudomonas species, and Salmonella species. Specific examples of infectious bacteria include but are not limited to Heliobacter pyloris, Borrelia burgdorferi, Legionella pneumophilia, Mycobacteria spp. (for example M. tuberculosis, M. avium, M. intracellilare, M. kansaii, M. gordonae), Staphylococcus aureus, Neisseria gonorrhoeae, Neisseria meningitidis, Listeria monocytogeners, Streptococcus pyogenes, (group A Streptococcus), Streptococcus agalactiae(Group B Streptococcus), Streptococcus (viridans group), Streptococcus faecalis, streptococcus bovis, Streptococcus (aenorobic spp.), Streptococcus pneumoniae, pathogenic Campylobacter spp., Enterococcus spp., Haemophilus influenzae, Bacillus anthracis, Corynebacterium diptheriae, Corynebacterium spp., Erysipelothrix rhusiopathie, Clostridium perfringens, Clostridium tetani, Enterobacter aerogenes, Klebsiella pneumoniae, Pasteurella multocida, Bacteroides spp., Fusobacterium nucleatum, Streptobacillus moniliformis, Treponema pallidum, Treponema pertenue, Leptospira, Rickettsia, and Actinomyces israelii.

Examples of viruses that cause infections in humans include but are not limited to Retroviridae (for example human deficiency viruses, such as HIV-1 (also referred to as HTLV-III), HIV-II, LAC or IDLV-III/LAV or HIV-III and other isolates such as HIV-LP, Picornaviridae (for example poliovirus, hepatitis A, enteroviruses, human Coxsackie viruses, rhinoviruses, echoviruses), Calciviridae (for example strains that cause gastroenteritis), Togaviridae (for example equine encephalitis viruses, rubella viruses), Flaviviridae (for example dengue viruses, encephalitis viruses, yellow fever viruses) Coronaviridae (for example coronaviruses), Rhabdoviridae (for example vesicular stomata viruses, rabies viruses), Filoviridae (for example Ebola viruses) Paramyxoviridae (for example parainfluenza viruses, mumps viruses, measles virus, respiratory syncytial virus), Orthomyxoviridae (for example influenza viruses), Bungaviridae (for example Hataan viruses, bunga viruses, phleoboviruses, and Nairo viruses), Arena viridae (hemorrhagic fever viruses), Reoviridae (for example reoviruses, orbiviruses, rotaviruses), Bimaviridae, Hepadnaviridae (hepatitis B virus), Parvoviridae (parvoviruses), Papovaviridae (papilloma viruses, polyoma viruses), Adenoviridae (adenoviruses), Herpeviridae (for example herpes simplex virus (HSV) I and II, varicella zoster virus, pox viruses) and Iridoviridae (for example African swine fever virus) and unclassified viruses (for example the etiologic agents of Spongiform encephalopathies, the agent of delta hepatitis, the agents of non-A, non-B hepatitis (class 1 enterally transmitted; class 2 parenterally transmitted such as Hepatitis C); Norwalk and related viruses and astroviruses).

Examples of fungi include Aspergillus spp., Coccidoides immitis, Cryptococcus neoformans, Candida albicans and other Candida spp., Blastomyces dermatidis, Histoplasma capsulatum, Chlamydia trachomatis, Nocardia spp., and Pneumocytis carinii.

Parasites include but are not limited to blood-borne and/or tissue parasites such as Babesia microti, Babesi divergans, Entomoeba histolytica, Giarda lamblia, Leishmania tropica, Leishmania spp., Leishmania braziliensis, Leishmania donovdni, Plasmodium falciparum, Plasmodium malariae, Plasmodium ovale, Plasmodium vivax, Toxoplasma gondii, Trypanosoma gambiense and Trypanosoma rhodesiense (African sleeping sickness), Trypanosoma cruzi (Chagus' disease) and Toxoplasma gondii, flat worms, and round worms.

As noted this invention further embraces the use of the subject conjugates in treating proliferative diseases such as cancers. Cancer is a condition of uncontrolled growth of cells which interferes with the normal functioning of bodily organs and systems. A subject that has a cancer is a subject having objectively measurable cancer cells present in the subjects' body. A subject at risk of developing cancer is a subject predisposed to develop a cancer, for example based on family history, genetic predisposition, subject exposed to radiation or other cancer-causing agent. Cancers which migrate from their original location and seed vital organs can eventually lead to the death of the subject through the functional deterioration of the affected organ. Hematopoietic cancers, such as leukemia, are able to out-compete the normal hematopoietic compartments in a subject thereby leading to hematopoietic failure (in the form of anemia, thrombocytopenia and neutropenia), ultimately causing death.

A metastasis is a region of cancer cells, distinct from the primary tumor location, resulting from the dissemination of cancer cells from the primary tumor to other parts of the body. At the time of diagnosis of the primary tumor mass, the subject may be monitored for the presence of metastases. Metastases are often detected through the sole or combined use of magnetic resonance imaging (MRI), computed tomography (CT), scans, blood and platelet counts, liver function studies, chest —X-rays and bone scans in addition to the monitoring of specific symptoms.

The compositions, protein conjugates and DNA vaccines of the invention can be used to treat a variety of cancers or subjects at risk of developing cancer, by the inclusion of a tumor-associated-antigen (TAA), or DNA encoding. This is an antigen expressed in a tumor cell. Examples of such cancers include breast, prostate, colon, blood cancers such as leukemia, chronic lymphocytic leukemia, and the like. The vaccination methods of the invention can be used to stimulate an immune response to treat a tumor by inhibiting or slowing the growth of the tumor or decreasing the size of the tumor. A tumor associated antigen can also be an antigen expressed predominantly by tumor cells but not exclusively.

Additional cancers include but are not limited to basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), lymphoma including Hodgkin's lymphoma and non-Hodgkin's lymphoma; melanoma; neuroblastoma; oral cavity cancer (for example lip, tongue, mouth and pharynx); ovarian cancer; pancreatic cancer; retinoblastoma; rhabdomyosarcoma; rectal cancer; cancer of the respiratory system; sarcoma; skin cancer; stomach cancer; testicular cancer; thyroid cancer; uterine cancer; cancer of the urinary system; as well as other carcinomas and sarcomas.

The compositions, protein conjugates, and DNA s of the invention can also be used to treat autoimmune diseases such as multiple sclerosis, rheumatoid arthritis, type 1 diabetes, psoriasis or other autoimmune disorders. Other autoimmune disease which potentially may be treated with the vaccines and immune adjuvants of the invention include Crohn's disease and other inflammatory bowel diseases such as ulcerative colitis, systemic lupus eythematosus (SLE), autoimmune encephalomyelitis, myasthenia gravis (MG), Hashimoto's thyroiditis, Goodpasture's syndrome, pemphigus, Graves disease, autoimmune hemolytic anemia, autoimmune thrombocytopenic purpura, scleroderma with anti-collagen antibodies, mixed connective tissue disease, polypyositis, pernicious anemia, idiopathic Addison's disease, autoimmune associated infertility, glomerulonephritis) for example crescentic glomerulonephritis, proliferative glomerulonephritis), bullous pemphigoid, Sjogren's syndrome, psoriatic arthritis, insulin resistance, autoimmune diabetes mellitus (type 1 diabetes mellitus; insulin dependent diabetes mellitus), autoimmune hepatitis, autoimmune hemophilia, autoimmune lymphoproliferative syndrome (ALPS), autoimmune hepatitis, autoimmune hemophilia, autoimmune lymphoproliferative syndrome, autoimmune uveoretinitis, and Guillain-Bare syndrome. Recently, arteriosclerosis and Alzheimer's disease have been recognized as autoimmune diseases. Thus, in this embodiment of the invention the antigen will be a self-antigen against which the host elicits an unwanted immune response that contributes to tissue destruction and the damage of normal tissues.

The compositions, protein conjugates and DNA vaccines of the invention can also be used to treat asthma and allergic and inflammatory diseases. Asthma is a disorder of the respiratory system characterized by inflammation and narrowing of the airways and increased reactivity of the airways to inhaled agents. Asthma is frequently although not exclusively associated with atopic or allergic symptoms. Allergy is acquired hypersensitivity to a substance (allergen). Allergic conditions include eczema, allergic rhinitis, or coryza, hay fever, bronchial asthma, urticaria, and food allergies and other atopic conditions. An allergen is a substance that can induce an allergic or asthmatic response in a susceptible subject. There are numerous allergens including pollens, insect venoms, animal dander, dust, fungal spores, and drugs.

Examples of natural and plant allergens include proteins specific to the following genera: Canine, Dermatophagoides, Felis, Ambrosia, Lotium, Cryptomeria, Alternaria, Alder, Alinus, Betula, Quercus, Olea, Artemisia, Plantago, Parietaria, Blatella, Apis, Cupressus, Juniperus, Thuya, Chamaecyparis, Periplanet, Agopyron, Secale, Triticum, Dactylis, Festuca, Poa, Avena, Holcus, Anthoxanthum, Arrhenatherum, Agrostis, Phleum, Phalaris, Paspalum, Sorghum, and Bromis.

It is understood that the compositions, protein conjugates and DNA vaccines of the invention can be combined with other therapies for treating the specific condition, e.g., infectious disease, cancer or autoimmune condition. For example in the case of cancer the inventive methods may be combined with chemotherapy or radiotherapy.

Methods of making compositions as vaccines are well known to those skilled in the art. The effective amounts of the protein conjugate or DNA can be determined empirically, but can be based on immunologically effective amounts in animal models. Factors to be considered include the antigenicity, the formulation, the route of administration, the number of immunizing doses to be administered, the physical condition, weight, and age of the individual, and the like. Such factors are well known to those skilled in the art and can be determined by those skilled in the art (see for example Paoletti and McInnes, eds., Vaccines, from Concept to Clinic: A Guide to the Development and Clinical Testing of Vaccines for Human Use CRC Press (1999). As disclosed herein it is understood that the subject DNAs or protein conjugates can be administered alone or in conjunction with other adjuvants.

The DNAs and protein conjugates of the invention can be administered locally or systemically by any method known in the art including but not limited to intramuscular, intravenous, intradermal, subcutaneous, intraperitoneal, intranasal, oral or other mucosal routes. Additional routes include intracranial (for example intracisternal, or intraventricular), intraorbital, ophthalmic, intracapsular, intraspinal, and topical administration. The adjuvants and vaccine compositions of the invention can be administered in a suitable, nontoxic pharmaceutical carrier, or can be formulated in microcapsules or a sustained release implant. The immunogenic compositions of the invention can be administered multiple times, if desired, in order o sustain the desired cellular immune response. The appropriate route, formulation, and immunization schedule can be determined by one skilled in the art.

In the methods of the invention, in some instances the antigen and a TLR/CD40 agonist conjugate may be administered separately or combined in the same formulation. In some instances it may be useful to include several antigens. These compositions may be administered separately or in combination in any order that achieve the desired synergistic enhancement of cellular immunity. Typically, these compositions are administered within a short time of one another, i.e. within about several hours of one another, more preferably within about a half hour.

In some instances, it may be beneficial to include a moiety in the conjugate or the DNA which facilitates affinity purification. Such moieties include relatively small molecules that do not interfere with the function of the polypeptides in the conjugate. Alternatively, the tags may be removable by cleavage. Examples of such tags include poly-histidine tags, hemagglutinin tags, maltase binding protein, lectins, glutathione-S transferase, avidin and the like. Other suitable affinity tags include FLAG, green fluorescent protein (GFP), myc, and the like.

The subject protein conjugates and DNAs can be administered with a physiologically acceptable carrier such as physiological saline. The composition may also include another carrier or excipient such as buffers, such as citrate, phosphate, acetate, and bicarbonate, amino acids, urea, alcohols, ascorbic acid, phospholipids, proteins such as serum albumin, ethylenediamine tetraacetic acid, sodium chloride or other salts, liposomes, mannitol, sorbitol, glycerol and the like. The agents of the invention can be formulated in various ways, according to the corresponding route of administration. For example, liquid formulations can be made for ingestion or injection, gels or procedures can be made for ingestion, inhalation, or topical application. Methods for making such formulations are well known and can be found in for example, “Remington's Pharmaceutical Sciences,” 18^{th} Ed., Mack Publishing Company, Easton Pa.

As noted the invention embraces DNA based vaccines. These DNAs may be administered as naked DNAs, or may be comprised in an expression vector. Furthermore, the subject nucleic acid sequences may be introduce into a cell of a graft prior to transplantation of the graft. This DNA preferably will be humanized to facilitate expression in a human subject.

The subject polypeptide conjugates may further include a “marker” or “reporter”. Examples of marker or reporter molecules include beta lactamase, chloramphenicol acetyltransferase, adenosine deaminase, aminoglycoside phosphotransferase, dihydrofolate reductase, hygromycin B-phosphotransferase, thymidine kinase, lacZ, and xanthine guanine phosphoribosyltransferase et al.

The subject nucleic acid constructs can be contained in any vector capable of directing its expression, for example a cell transduced with the vector. The inventors used a baculovirus vector as they have much experience using this vector. Other vectors which may be used include T7 based vectors for use in bacteria, yeast expression vectors, mammalian expression vectors, viral expression vectors, and the like. Viral vectors include retroviral, adenoviral, adeno-associated vectors, herpes virus, simian virus 40, and bovine papilloma virus vectors.

Prokaryotic and eukaryotic cells that can be used to facilitate expression of the subject polypeptide conjugates include by way of example microbia, plant and animal cells, e.g., prokaryotes such as Escherichia coli, Bacillus subtilis, and the like, insect cells such as Sf21 cells, yeast cells such as Saccharomyces, Candida, Kluyveromyces, Schizzosaccharomyces, and Pichia, and mammalian cells such as COS, HEK293, CHO, BHK, NIH 3T3, HeLa, and the like. One skilled in the art can readily select appropriate components for a particular expression system, including expression vector, promoters, selectable markers, and the like suitable for a desired cell or organism. The selection and use of various expression systems can be found for example in Ausubel et al., “Current Protocols in Molecular Biology, John Wiley and Sons, New York, N.Y. (1993); and Pouwels et al., Cloning Vectors: A Laboratory Manual”: 1985 Suppl. 1987). Also provided are eukaryotic cells that contain and express the subject DNA constructs.

In the case of cell transplants, the cells expressing such DNA conjugate can be administered either by an implantation procedure or with a catheter-mediated injection procedure through the blood vessel wall. In some cases, the cells may be administered by release into the vasculature, from which the cells subsequently are distributed by the blood stream and/or migrate into the surrounding tissue.

The subject polypeptide conjugates or the DNA constructs typically contain or encode an anti-CD40 antibody or fragment thereof that specifically binds CD40, preferably murine or human CD40 or another CD40 agonist such as a CD40L polypeptide or fragment, mutant or conjugate containing. As used herein, the term “antibody” is used in its broadest sense to include polyclonal and monoclonal antibodies, as well as antigen binding fragments thereof. This includes Fab, F(ab′)2, Fd and Fv fragments.

In addition the term “antibody” includes naturally antibodies as well as non-naturally occurring antibodies such as single chain antibodies, chimeric antibodies, bifunctional and humanized antibodies. Preferred for use in the invention are chimeric, humanized and fully human antibodies. Methods for synthesis of chimeric, humanized, CDR-grafted, single chain and bifunctional antibodies are well known to those skilled in the art. In addition, antibodies specific to CD40 are widely known and available and can be made by immunization of a suitable host with a CD40 antigen, preferably human CD40.

It is understood that modifications which do not substantially affect the activity of the various embodiments of this invention are also provided within the definition of the invention provided herein. Accordingly, the following examples are intended to illustrate but not limit the present invention.

### EXAMPLES

### Construction of Vaccine for Eliciting Cellular Immunity Against HIV

Conjugate vaccines prepared by the methods described supra were constructed for immunization against HIV Gag. The AAD transgenic mouse expresses a mutant HLA A2 molecule that contains the alpha3 domain of H-2D and thus is able to bind mouse CD8 (Newberg et al., J Immunol. 156:2473 (1996); Kan-Mitchell et al., J Immunol. 172:5249 (2004)) Using HLA A2 tetramers, HLA A2/peptide specific T cells generated in this mouse can be easily detected (Bullock et al., J Immunol. 170:1822 (2003)). The SLYNTVATL epitope (S19) is a dominant CD8 epitope from HIV p21Gag (Kan-Mitchell et al. (Id). Therefore, following immunization of AAD mice, the HLA A2/SLYNTVATL specific CD8+ T cell response is analyzed by tetramer, intracellular (IC) IFNgamma (Ahonen et al. (Id)), and CD107a staining (for cytotoxic function (Betts et al., J Immunol Methods, 281:65 (2003), as previously described. The Gag-specific CD4 response will be similarly monitored by IC IFNgamma staining. Gag specific antibody titer and isotypes in the serum will be monitored by ELISA as previously described. (Wille-Reece et al., J Immunol. 174:7676 (2005)).

Mice are then immunized either with recombinant protein conjugate by IP or SC, or by DNA immunization injected IM. As a positive control, separate mice are immunized with protein, anti-CD40 antibody, and purified flagellin or 3M012 as shown in FIG. 1. The primary CD4 and CD8+ T cell responses to combined TLR/CD40-agonist immunization peaks in the blood seven days after immunization. Based on our extensive experience in monitoring antigen specific T cells following immunization; the blood is the most sensitive site for detecting antigen specific T cells following immunization; the blood is also the most sensitive site for detecting T cells, due mostly to the extremely low background of either tetramer and IC IFNgamma staining of the cells in the blood compared to cells from the lymph node (LN) or spleen (unpublished observations). This is an advantage because immunized mice can be monitored continuously for both T cell and antibody responses by tail bleeding at different time points. The primary T cell responses will be monitored in the peripheral blood between days 5 and 12 in order to determine both the magnitude and time course of the primary response. Serum will be taken on days 10 and 25 after primary immunization to determine antigen specific antibody titers. At least 60 days after primary immunization, the secondary immune responses are analyzed by boosting the mice in the same fashion that they were initially immunized. The secondary responses will be determined in the same fashion as the primary immune response except that the T cell responses in the blood will be analyzed 5 days after boosting (see FIG. 1C). The secondary antibody responses are assayed in the serum 7 days after boosting.

Immunity Following Parenteral or Mucosal Vaccination

There is increasing evidence that IP or Sc routes of immunization do not effectively generate mucosal immunity (Lajeunesse et al., Adv Exp Med Biol 549:13 (2004) surfaces (nasal, oral, rectal, vaginal) generates long term immunity that specifically localizes to mucosal tissues through the host. For example, in numerous animal model systems, immunization through the nasal mucosa was found to provide protective immunity against challenge through the vaginal mucosa whereas SC immunization did not (Leavell et al., Vaccine 23:996 (2005); Shanley et al., Vaccine 23:1471 (2005); Devito et al., J Immunol 173:7078 (2004); Kwant A and Rosenthal Vaccine 22:3098 (2004) and Belyakov et al., J Immunol. 164:725 (2000). Therefore, the site in which immunity is initiated may be as important as the actual magnitude of the response. Because the genital mucosa is the primary site of entry for HIV infection, it is critical to determine whether the subject DNA and protein based vaccines are able to generate and maintain mucosal immunity.

Methods for Conferring Mucosal and Non-Mucosal Immunity

Mice are challenged with the protein or the DNA vaccine as described above and the T cell response elicited from SC and IF immunization is compared to that generated after mucosal immunization. Mice are immunized either nasally or rectally, as previously described for protein/peptide immunization (Belyakov et al., J Immunol 174:725 (2000)). Following immunization, T cell and antibody responses are assessed in both the blood as well as in the nasal and vaginal mucosa as described (Shanley et al., Vaccine 23:996 (2005); Devito et al., J Immunol 173:7078 (2004); Kwant et al., Vaccine 22:3098 (2004); Belyakov et al., Proc Natl Acad Sci USA 95:1709 (1998); and Belyakov et al., Proc Natl Acad Sci, USA 96:4512 (1999). 60 days following primary immunization, the mice are again boosted with a second dose of vaccine. In particular, mice originally immunized via a mucosal route are split into two groups, one boosted mucosally and the other boosted parenterally. This will determine whether mucosal immunity is maintained by parenteral boosting. The CD4, CD8, and antibody responses are monitored as described below.

Anticipated Results

It is anticipated that immunization with a protein or DNA conjugate according to the invention (TLR/CD40-agonist vaccine) will dramatically enhance all arms of adaptive immunity. One of the advantages of a conjugate vaccine as described herein is that the antigen can be delivered with greater efficiency to dendritic cells while simultaneously activating the DC via both CD40 and TLR. Therefore, it is anticipated that immunization with the protein vaccine containing flagellin will be even more effective at generating immunity than control injections of a non-conjugate vaccine. It is also anticipated that both parenteral and mucosal challenge will generate potent immunity. Consistent with other mucosal viral vaccine vaccines it is anticipated that mucosal challenge will be superior to parenteral challenge at eliciting mucosal specific T and B cell immunity; i.e., T cell homing and IgA production within the mucosal tissue. It is believed that the results of the afore-described experiments will reveal that the initiation of immunity within a mucosal site directs its function to the mucosa and that this mucosal preference will be maintained independent of future boosting. Therefore, following primary mucosal immunization, it is envisioned that boosting by any means will enhance both mucosal and peripheral T and B cell immune memory. TLR5 expression has been observed to be high in mucosal tissues such as the intestines (Schmausser et al., Clin Exp Immunol 136:521 (2004); Maaser et al., J Immunol. 172:5056 (2004)) and therefore we anticipate that protein and DNA immunization containing flagellin will demonstrate an advantage over TLR agonists that target other TLRs not as highly expressed in mucosal tissues.

A further advantage of DNA immunization according to the invention is that it avoids the problems sometimes associated with producing high yields of protein. However, protein vaccines are advantageous in that they possess a relatively short half-life in vivo. After DNA immunization because of potential unintended effects on the immune system we will further titrate DNA immunizations and determine by ELISA the duration of the protein production following immunization as well as its effects on the immune response.

The data discussed herein supports the efficacy of a combined TLR/CD40-agonist vaccine for promoting protective immunity against a target. Following priming with a combined TLR/CD40-agonist immunization, secondary challenge with vaccinia virus elicited a robust secondary CD8+ T cell response, comprising 30-60% of all circulating CD8+ T cells, even in CD4 deficient or depleted hosts. Furthermore, viral titers were similarly reduced in mice previously immunized with a combined TLR/CD40-agonist vaccination, whether they were CD4 depleted or not. The results herein therefore suggest that the subject DNA and protein conjugate vaccines will confer immunity even in immunocompromised subjects (HIV CD4 deficient subjects). Therefore, the invention is particularly well suited in promoting cellular immunity in lymphopenic patients (with respect to CD4 cells) such as those infected with HIV or a cancer or other disease that affects CD4 cells.

Testing for Protective Immunity Upon Challenge

The degree of protective immunity conferred on the host may be confirmed in an animal model. Particularly, female AAD mice, immunized as described above may be challenged with 5 million pfu of VVgag either IP, nasally, or rectally as previously described. 5-7 days after viral challenge, ovaries are removed, homogenized, sonicated, and measured for viral titers by plaque assay (Kedl et al., J Exp Med 192:1105 (2000); Belyakov et al., Proc Natl Acad Sci, USA 96:4512 (1999). CD4 and CD8+ T cell responses in the blood are monitored in the virally challenged mice in order to correlate immunologic endpoints with efficacy. Naïve mice and mice previously challenged with virus, as negative and positive controls for protective immunity, respectively, are challenged with Vvgag and viral titers measured.

Protective Immunity in wt and CD4 Deficient Hosts

The afore-discussed data suggests that even in CD4 deficient hosts, this form of vaccination generates competent CD8+ T cell memory. (See FIG. 2) This is significant both in the context of a prophylactic and a therapeutic HIV vaccine given the chronic drug use often associated with persons at risk. Therefore, the degree of protective immunity observed in CD4 and wt deficient hosts is also determined in an appropriate animal model.

ADD mice are bred on the class II knockout mouse background to produce mice deficient in CD4 cells but able to mount an HLA-A2/HIV Gag specific CD8+ T cell response. These mice are immunized mucosally or parenterally, challenged with Vvag, and the degree of mucosal and systemic protective immunity determined and compared to wt (wild type) mice. T cell responses in peripheral blood are again simultaneously monitored to correlate the expansion of CD8+ T cells in the CD4 deficient host with protective immunity. Preliminary results have shown that CD4 deficient mice have an approximately 2 fold reduction in CD8+ T cell numbers but not a significant reduction in protective immunity against viral challenge. (FIG. 2). Therefore, it is anticipated that the T cell responses for CD4 deficient and wt mice should be similar.

It is also anticipated based on these results that the present invention will provide novel methods of treatment of diseases wherein enhance cellular immunity is a desired therapeutic outcome, in particular chronic and debilitating human diseases such as cancer and other proliferative diseases, infectious diseases, autoimmunity, allergic conditions and inflammatory conditions such as arteriosclerosis. The invention is exemplified in the context of an HIV vaccine (protein or DNA conjugate) since this is a disease wherein enhanced cellular immunity will be required for an effective vaccine and it is further a disease wherein CD4 cells are depleted thus illustrating the efficacy of the subject methods for treating diseases wherein CD4 cells are depleted or impaired. However, the invention broadly encompasses the use of the DNA and protein conjugates of the invention for treating or prophylaxis of any disease wherein enhanced antigen specific cellular immunity is desirable including by way of example, cancer, allergy, inflammatory diseases, infection, and autoimmunity. Examples thereof are identified herein.

It is to be understood that the invention is not limited to the embodiments listed hereinabove and the right is reserved to the illustrated embodiments and all modifications coming within the scope of the following claims.

The various references to journals, patents, and other publications which are cited herein comprise the state of the art and are incorporated by reference as though fully set forth.

1. A nucleic acid construct comprising:
(i) at least one nucleic acid sequence encoding an agonist of CD40;
(ii) optionally a nucleic acid sequence encoding a desired antigen; and
(iii) a nucleic acid sequence encoding an agonist of at least one toll like receptor (TLR) selected from TLR1, TLR2, TLR3, TLR4, TLR5, TLR6, TLR7, TLR8, TLR9, TLR10 and/or TLR11; and
wherein said sequences (i), (ii) and (iii) are operably linked to the same or different transcription regulatory sequences and further wherein said sequences (i), (ii) and (iii) are optionally separated by a linker sequence and/or an IRES.

2. The nucleic acid construct of claim 1 wherein the polypeptide TLR agonist is a TLR5 or TLR11 agonist.

3. The nucleic acid construct of claim 1 wherein the TLR agonist is a flagellin or a prolifin-like TLR agonist molecule.

4. The nucleic acid construct of claim 3 wherein the TLR agonist is a flagellin or a fragment or variant thereof that stimulates TLR5.

5. The nucleic acid construct of claim 1 wherein the CD40 agonist is an anti-CD40 antibody or antibody fragment, CD40 binding aptamer or soluble CD40L, or a fragment, polymer or a conjugate containing.

6. The nucleic acid construct of claim 5 wherein said antibody is a chimeric immunoglobulin.

7. The nucleic acid construct of claim 5 wherein said antibody is a humanized immunoglobulin.

8. The nucleic acid construct of claim 5 wherein said antibody is a human immunoglobulin.

9. The nucleic acid construct of claim 5 wherein said antibody is a single chain immunoglobulin.

10. The nucleic acid construct of claim 5 wherein said antibody comprises human heavy and light chain constant regions.

11. The nucleic acid construct of claim 5 wherein said antibody is selected from the group consisting of an IgG1, IgG2, IgG3 and an IgG4.

12. The nucleic acid construct of claim 5 wherein said antibody is encoded by an immunoglobulin light chain encoding nucleic acid sequence and an immunoglobulin heavy chain encoding nucleic acid sequence which are operably linked to the same promoter.

13. The nucleic acid construct of claim 12 wherein said immunoglobulin light chain and immunoglobulin heavy chain sequences are intervened by an IRES.

14. The nucleic acid construct of claim 1 wherein said optional antigen sequence (ii) encodes a viral, bacterial, fungal, or parasitic antigen.

15. The nucleic acid construct of claim 1 wherein said sequence (ii) encodes a human antigen.

16. The nucleic acid construct of claim 15 wherein said human antigen is a cancer antigen, autoantigen or other human antigen the expression of which correlates or is involved in a chronic human disease.

17. The nucleic acid construct of claim 14 wherein said viral antigen is specific to a virus selected from the group consisting of HIV, herpes, papillomavirus, ebola, picorna, enterovirus, measles virus, mumps virus, bird flu virus, rabies virus, VSV, dengue virus, hepatitis virus, rhinovirus, yellow fever virus, bunga virus, polyoma virus, coronavirus, rubella virus, echovirus, pox virus, varicella zoster, African swine fever virus, influenza virus and parainfluenza virus.

18. The nucleic acid construct of claim 5 wherein said flagellin is a bacterial flagellin or a variant or fragment that activates TLR5.

19. The nucleic acid construct of claim 14 wherein said bacterial antigen is derived from a bacterium selected from the group consisting of Salmonella, Escherichia, Pseudomonas, Bacillus, Vibrio, Campylobacter, Heliobacter, Erwinia, Borrelia, Pelobacter, Clostridium, Serratia, Xanothomonas, Yersinia, Burkholdia, Listeria, Shigella, Pasteurella, Enterobacter, Corynebacterium and Streptococcus.

20. The nucleic acid construct of claim 14 wherein said parasite antigen is derived from a parasite selected from Babesia, Entomoeba, Leishmania, Plasmodium, Trypanosoma, Toxoplasma, Giarda, flat worms and round worms.

21. The nucleic acid construct of claim 14 wherein said fungal antigen is derived from a fungi selected from the group consisting of Aspergillus, Coccidoides, Cryptococcus, Candida Nocardia, Pneumocystis, and Chlamydia.

22. The nucleic acid construct of claim 3 wherein the flagellin is derived from Salmonella minnesota.

23. The nucleic acid construct of claim 22 wherein said flagellin has the amino acid sequence encoded by the nucleic acid contained in SEQ ID NO:1 or a sequence at least 90% identical thereto.

24. The nucleic acid construct of claim 1 wherein the antigen is a cancer antigen expressed by a human cancer selected from the group consisting of a CD40 expressing cancer cell, prostate cancer, pancreatic cancer, brain cancer, lung cancer (small or large cell), bone cancer, stomach cancer, liver cancer, breast cancer, ovarian cancer, testicular cancer, skin cancer, lymphoma, leukemia, colon cancer, thyroid cancer, cervical cancer, head and neck cancer, sarcoma, glial cancer, and gall bladder cancer

25. The nucleic acid construct of claim 1 wherein the antigen is an autoantigen the expression of which correlates to an autoimmune disease.

26. An expression vector containing a nucleic acid construct according to claim 1.

27. The expression vector of claim 26 which is selected from a plasmid, recombinant virus, and episomal vector.

28. A recombinant host cell or non-human animal which expresses a nucleic acid construct according to claim 1.

29. The recombinant host cell of claim 28 which is selected from bacterial cell, yeast cell, mammalian cell, insect cells, avian cell and amphibian cell.

30. The recombinant host cell of claim 28 which is a human cell.

31. A protein conjugate that results upon expression of the nucleic acid construct according to claim 1.

32. The protein conjugate of claim 31 which comprises an anti-CD40 antibody, flagellin or a fragment thereof that stimulates TLR5, and an antigen the expression of which correlates to a disease condition.

33. The protein conjugate of claim 32 wherein said diseases is selected from cancer, allergy, an autoimmune disease, an infectious disease and an inflammatory condition.

34. The protein conjugate of claim 33 which comprises an HIV antigen.

35. The protein conjugate of claim 34 wherein the HIV antigen is Gag, Pol or Env.

36. A method for eliciting an antigen specific cellular immune response by administering a nucleic acid construct according to claim 1 or a vector or host cell containing said nucleic acid construct.

37. The method of claim 36 wherein said administering results in a least one of the following:
(i) enhanced primary and memory CD8+ T cell responses relative to the administration of a DNA encoding only a CD40 agonist or TLR agonist;
(ii) induces exponential expansion of antigen specific CD8+ T cells; and
(iii) generates a protective immune response in a CD4 deficient host comparable to a normal (non-CD4 deficient) host

38. The method of claim 36 wherein the antigen is selected from a viral antigen, bacterial antigen, fungal antigen, autoantigen, allergen, and cancer antigen.

39. The method of claim 37 wherein the antigen is a HIV antigen.

40. The method of claim 39 wherein the HIV antigen is gag, pol or env.

41. The method of claim 38 wherein the antigen is an antigen expressed by a human tumor.

42. A method for eliciting an antigen specific cellular immune response in a subject in need thereof comprising administering a polypeptide conjugate comprising at least one CD40 agonist, at least one polypeptide TLR agonist and optionally at least one antigen the expression of which is correlated to a specific disease.

43. The method of claim 42 wherein the CD40 agonist is an anti-CD40 antibody or a soluble CD40L or fragment or conjugate containing.

44. The method of claim 42 wherein the TLR agonist is flagellin or a fragment thereof that induces TLR5.

45. The method of claim 42 wherein the disease is selected from cancer, allergy, inflammatory disease, infectious disease and an autoimmune disease.

46. The method of claim 44 wherein the infectious disease is caused by a virus, bacterium, fungus, or parasite.

47. The method of claim 45 wherein the virus is HIV.

48. The method of claim 42 wherein said administration results in at least one of the following:
(i) elicits substantially enhanced primary and memory CD8+ T cell responses relative to the administration of the CD40 agonist or the TLR agonist alone;
(ii) induces exponential expansion of antigen specific CD8+ T cells; and
(iii) generates a protective immune response in a CD4 deficient host that is comparable to a normal (non-CD4 deficient) host.

49. The method of claim 48 which is used to treat a viral infection or cancer.

50. The method of claim 36 or 42 wherein the protein conjugate is administered mucosally.

51. The method of claim 49 wherein mucosal delivery includes oral, intranasal, rectal and vaginal delivery methods.

52. The method of claim 42 which is used to treat a subject with a condition or genetic defect associated with impaired or depleted CD4+ cells.
